(19) United States
(12) Patent Application Publication
(10) Pub. No.: US 2022/0160748 A1
(43) Pub. Date: May 26, 2022

(54) CONJUGATES OF S-ANTIGEN TRANSPORT INHIBITING OLIGONUCLEOTIDE POLYMERS HAVING ENHANCED LIVER TARGETING

(71) Applicant: Aligos Therapeutics, Inc., South San Francisco, CA (US)

(72) Inventors: Leonid Beigelman, San Mateo, CA (US); Megan Elizabeth Fitzgerald, San Francisco, CA (US); Rajendra Pandey, South San Francisco, CA (US); Jin Hong, Brisbane, CA (US); Vivek Kumar Rajwanshi, Cupertino, CA (US); David Bernard Smith, San Mateo, CA (US); Lawrence M. Blatt, Healdsburg, CA (US)

(21) Appl. No.: 17/455,528

(22) Filed: Nov. 18, 2021

Related U.S. Application Data
(60) Provisional application No. 63/116,368, filed on Nov. 20, 2020.

(51) Int. Cl. A61K 31/7088; A61K 47/54; A61P 1/16

(52) U.S. Cl. CPC A61K 31/7088; A61K 47/549; A61K 47/543; A61P 1/16; A61K 47/542; A61K 47/547; A61K 47/545

(57) ABSTRACT

Disclosed herein is an oligonucleotide conjugate or complex thereof, comprising a modified oligonucleotide having sequence independent antiviral activity against hepatitis B, a liver targeting agent, and a linker connecting the liver targeting agent to a 3′ end and/or a 5′ end of the modified oligonucleotide or complex thereof. The oligonucleotide conjugate may be incorporated into a pharmaceutical composition for treating a liver disease or disorder such as hepatitis B.

### INCORPORATION BY REFERENCE TO PRIORITY APPLICATION

This application claims priority to U.S. Provisional Application Ser. No. 63/116,368, filed Nov. 20, 2020, which is hereby incorporated herein by reference in its entirety.

### REFERENCE TO SEQUENCE LISTING

The present application is being filed along with a Sequence Listing in electronic format. The Sequence Listing is provided as a file entitled SeqList_ALIG060A2.TXT, which was created and last modified on Nov. 24, 2021 and is approximately 213 kilobytes in size. The information in the electronic Sequence Listing is hereby incorporated by reference in its entirety.

### BACKGROUND

### Field

This application relates to STOPS™ antiviral compounds that are conjugates of S-antigen transport inhibiting oligonucleotide polymers having enhanced liver targeting, processes for making them, and methods of using them to treat liver diseases and conditions.

### Description

A number of patent applications filed in the early 2000s disclosed phosphorothioated antiviral oligonucleotides that exert their antiviral activity by what has been termed a non-sequence dependent mode of action. See, e.g., U.S. Pat. Nos. 7,358,068; 8,008,269; 8,008,270 and 8,067,385. The structures of certain specific compounds were identified along with various options as potential areas for future experimentation. The effect of structural modifications on antiviral activity was the subject of subsequent active research over the course of a number of years. See A. Vaillant, “Nucleic acid polymers: Broad spectrum antiviral activity, antiviral mechanisms and optimization for the treatment of hepatitis B and hepatitis D infection”, Antiviral Research 133, 32-40 (2016). These efforts resulted in the identification of the compound known to those skilled in the art as REP 2139, a phosphorothioated 40-mer having repeating adenosine-cytidine (AC) units with 5-methylation of all cytosines and 2′-O methyl modification of all riboses, along with the compound known as its clinical progenitor, REP 2055. See I. Roehl et al., “Nucleic Acid Polymers with Accelerated Plasma and Tissue Clearance for Chronic Hepatitis B Therapy”, Molecular Therapy: Nucleic Acids Vol. 8, 1-12 (2017). The authors of that publication indicated that the structural features of these compounds had been optimized for the treatment of hepatitis B and hepatitis D. See also A. Vaillant, “Nucleic acid polymers: Broad spectrum antiviral activity, antiviral mechanisms and optimization for the treatment of hepatitis B and hepatitis D infection”, Antiviral Research 133 (2016) 32-40. According to these authors and related literature, such compounds preserve antiviral activity against hepatitis B while preventing recognition by the innate immune response to allow their safe use with immunotherapies such as pegylated interferon.

More recently, a number of STOPS™ antiviral compounds have been developed. See WO 2020/097342, WO 2021/119325, US 2020/0147124 and U.S. Pat. No. 11,166,976. These compounds are antiviral oligonucleotides that can be at least partially phosphorothioated and exert their antiviral activity by a sequence independent mode of action that is not based on hybridizing to a complementary sequence. The WO 2020/097342, WO 2021/119325, US 2020/0147124 and U.S. Pat. No. 11,166,976 publications include data showing that compounds containing various specified monomers provide significantly improved sequence independent antiviral activity against hepatitis B, in many cases >2-fold or even >5-fold higher potency than REP 2139.

The STOPS™ antiviral compounds described in WO 2020/097342, WO 2021/119325, US 2020/0147124 and U.S. Pat. No. 11,166,976 represent a significant advance in the art. However, there remains a long-felt need for more effective compounds in this class, and particularly for antiviral oligonucleotides having enhanced targeting to the liver for the treatments of various liver diseases and disorders, such as HBV.

### SUMMARY

Various embodiments provide an oligonucleotide conjugate or complex thereof, comprising:
a modified oligonucleotide of 30 to 50 nucleotides in length and having sequence independent antiviral activity against hepatitis B;
a liver targeting agent; and
a linker connecting the liver targeting agent to a 3′ end and/or a 5′ end of the modified oligonucleotide or a complex thereof;
wherein:
each liver targeting agent is independently GalNac1, GalNac2, GalNac3, GalNac4, GalNac5, GalNac6, a cholesterol, a tocopherol, a C₁₂₋₂₀ alkyl ester or a C₁₂₋₂₀ alkenyl ester;
each linker is independently a C₄₋₈ alkyl, -po-(Nuc-po)ₓ-, -po-O-(CH₂CH₂O)ₘ-po- or -po[-O-(CH₂CH₂O)ₘ-po-O-(CH₂CH₂O)ₙ]-po-;
each Nuc in -po-(Nuc-po)ₓ- is independently an A nucleotide or a C nucleotide;
each A nucleotide in -po-(Nuc-po)ₓ- is independently adenosine, 2′-O-methyladenosine, or deoxyadenosine;
each C nucleotide in -po-(Nuc-po)ₓ- is independently cytidine, 2′-O-methylcytidine, a locked/bridged cytidine, a locked/bridged methylcytidine, ribose cytidine, or deoxycytidine;
po is phosphodiester;
each x is independently an integer in the range of 1 to 5; and
m and n are each independently an integer in the range of 2 to 10.

In various embodiments, the liver targeting agent and the linker of the oligonucleotide conjugate or complex thereof are selected to provide improved targeting of the modified oligonucleotide to the liver of a mouse, non-human primate, human, or other mammalian subject to which the oligonucleotide conjugate or complex thereof is administered, wherein the improved targeting is determined by comparison to a corresponding modified oligonucleotide conjugate that lacks the linker, at a 6-hour, 24-hour, or 48-hour timepoint following subcutaneous administration to a mouse, non-human primate, human, or other mammalian subject at a dosage of about 0.05, 0.1, 0.15, 0.25, 0.5, 0.75, 1, 2, 3, 4, 5, 6, 7, 8, 9, 10, 11, 12, 13, 14 or 15 mg/kg, or any range that is defined by two of the aforementioned values as endpoints.

Various embodiments provide a pharmaceutical composition for treating a liver disease or disorder in a subject, comprising:
a pharmaceutically acceptable carrier, diluent, excipient or combination thereof; and
a therapeutically effective amount of an oligonucleotide conjugate or complex thereof as described herein.

Various embodiments provide a method of treating a liver disease or disorder, comprising administering an effective amount of the oligonucleotide conjugate or complex thereof as described herein, or a pharmaceutical composition as described herein, to a subject in need thereof.

In various embodiments, the liver disease or disorder is hepatitis B, hepatitis D or a liver cirrhosis that is developed because of a Hepatitis B infection.

These and other embodiments are described in greater detail below.

### BRIEF DESCRIPTION OF THE DRAWINGS

FIGS. 1A, 1B, and 1C schematically depict non-limiting examples of oligonucleotide conjugates comprising modified oligonucleotides, linkers, and liver targeting agents.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein the term “oligonucleotide” (or “oligo”) has its usual meaning as understood by those skilled in the art and thus refers to a class of compounds that includes oligodeoxynucleotides, oligodeoxyribonucleotides and oligoribonucleotides. Thus, “oligonucleotide” refers to an oligomer or polymer of ribonucleic acid (RNA) or deoxyribonucleic acid (DNA) or mimetics thereof, including reference to oligonucleotides composed of naturally-occurring nucleobases, sugars and phosphodiester (PO) internucleoside (backbone) linkages as well as “modified” or substituted oligonucleotides having non-naturally-occurring portions which function similarly. Thus, the term “modified” (or “substituted”) oligonucleotide has its usual meaning as understood by those skilled in the art and includes oligonucleotides having one or more of various modifications, e.g., stabilizing modifications, and thus can include at least one modification in the internucleoside linkage and/or on the ribose, and/or on the base. For example, a modified oligonucleotide can include modifications at the 2′-position of the ribose, acyclic nucleotide analogs, base modifications such as methylation (e.g., 5-methylcytidine), phosphorothioated (PS) linkages, phosphorodithioate linkages, methylphosphonate linkages, phosphoramidate or thiophosphoramidate linkages that connect to the sugar ring via sulfur or nitrogen, and/or other modifications. Thus, a modified oligonucleotide can include one or more phosphorothioated (PS) linkages, instead of or in addition to PO linkages. Like unmodified oligonucleotides, modified oligonucleotides that include such PS linkages are considered to be in the same class of compounds because even though the PS linkage contains a phosphorous-sulfur double bond instead of the phosphorous-oxygen double bond of a PO linkage, both PS and PO linkages connect to the sugar rings through oxygen atoms.

As used herein the term “oligonucleotide conjugate” has its usual meaning as understood by those skilled in the art and thus refers to an oligonucleotide that is attached to a ligand, such as a targeting agent. Such attachment may be direct (e.g., via a covalent bond) or indirect (e.g., via a linker group connecting the oligonucleotide to the ligand).

As used herein the term “linker” has its usual meaning as understood by those skilled in the art and thus refers to a component that connects a molecule or functional group to another. The linkers described herein may be used as spacers to control the overall distance from the modified oligonucleotide to the ligand(s) to which it is conjugated. The linkers described herein may also be selected on the basis of the manner in which they influence the physicochemical properties, solubility properties, in-vivo and other properties of the conjugates in which they are included. For example, the linker can be a covalent bond, a cleavable linker prone to enzymatic or chemical cleavage, or a non-cleavable linker.

As used herein the term “targeting” has its usual meaning as understood by those skilled in the art and thus refers to enhancing localization or delivery of a biologically active agent to a particular cell type, tissue type and/or bodily organ. As used herein the term “targeting agent” has its usual meaning as understood by those skilled in the art and thus refers to a ligand having affinity for a particular cell type, tissue type and/or organ. For example, a liver targeting agent is a targeting agent that has an affinity for the liver. Appropriate attachment of an oligonucleotide to such a liver targeting agent (e.g., via a linker) results in an oligonucleotide conjugate that provides targeted delivery of the oligonucleotide to the liver of a subject to which the oligonucleotide conjugate is administered. Such targeted delivery enhances the concentration of the oligonucleotide in the target organ as compared to the corresponding unconjugated oligonucleotide, and/or as compared to the corresponding oligonucleotide conjugate that lacks the linker.

As used herein in the context of oligonucleotides or other materials, the term “antiviral” has its usual meaning as understood by those skilled in the art and thus includes an effect of the presence of the oligonucleotide and/or other agent that inhibits production of viral particles, typically by reducing the number of infectious viral particles formed in a system otherwise suitable for formation of infectious viral particles for at least one virus. An antiviral oligonucleotide may have antiviral activity against multiple different viruses, e.g., both HBV and HDV.

As used herein in the context of modified oligonucleotides, the term “phosphorothioated” oligonucleotide has its usual meaning as understood by those skilled in the art and thus refers to a modified oligonucleotide in which all of the phosphodiester internucleoside linkages have been replaced by phosphorothioate linkages. Those skilled in the art thus understand that the term “phosphorothioated” oligonucleotide is synonymous with “fully phosphorothioated” oligonucleotide. A phosphorothioated oligonucleotide (or a sequence of phosphorothioated oligonucleotides within a partially phosphorothioated oligonucleotide) can be modified analogously, including (for example) by replacing one or more phosphorothioated internucleoside linkages by phosphodiester linkages. Thus, the term “modified phosphorothioated” oligonucleotide refers to a phosphorothioated oligonucleotide that has been modified in the manner analogous to that described herein with respect to oligonucleotides, e.g., by replacing a phosphorothioated linkage with a modified linkage such as phosphodiester, phosphorodithioate, methylphosphonate, diphosphorothioate, 5′-phosphoramidate, 3′,5′-phosphordiamidate, 5′-thiophosphoramidate, 3′,5′-thiophosphordiamidate or diphosphodiester. An at least partially phosphorothioated sequence of a modified oligonucleotide can be modified similarly, and thus, for example, can be modified to contain a non-phosphorothioated linkage such as phosphodiester, phosphorodithioate, methylphosphonate, diphosphorothioate 5′-phosphoramidate, 3′,5′-phosphordiamidate, 5′-thiophosphoramidate, 3′,5′-thiophosphordiamidate or diphosphodiester. In the context of describing modifications to a phosphorothioated oligonucleotide, or to an at least partially phosphorothioated sequence of a modified oligonucleotide, modification by inclusion of a phosphodiester linkage may be considered to result in a modified phosphorothioated oligonucleotide, or to a modified phosphorothioated sequence, respectively. Analogously, in the context of describing modifications to an oligonucleotide, or to an at least partially phosphodiesterified sequence of a modified oligonucleotide, the inclusion of a phosphorothioated linkage may be considered to result in a modified oligonucleotide or a modified phosphodiesterified sequence, respectively.

The term “sequence independent” antiviral activity has its usual meaning as understood by those skilled in the art and thus refers to an antiviral activity of an oligonucleotide (e.g., a modified oligonucleotide) that is not based on hybridizing to a complementary sequence. Such oligonucleotides are typically 30 to 50 nucleotides in length, e.g., about 40 nucleotides. Methods for determining whether the antiviral activity of an oligonucleotide is sequence independent are known to those skilled in the art and include the tests for determining if an oligonucleotide acts predominantly by a non-sequence complementary mode of action as disclosed in Example 10 of U.S. Pat. Nos. 7,358,068; 8,008,269; 8,008,270 and 8,067,385, which is hereby incorporated herein by reference and particularly for the purpose of describing such tests.

The term “oligonucleotide inhibitor” refers to an oligonucleotide (such as siRNA or antisense oligonucleotide) having an antiviral activity that is based on hybridizing to a complementary sequence. Such an oligonucleotide inhibitor is typically 7 to 26 nucleotides in length and at least partially inhibits production by a cell of an RNA or protein to which it is targeted, by hybridizing to the target RNA through base pairing. For example, an siRNA oligonucleotide inhibitor may cause silencing of a gene that encodes an mRNA and thus decrease the products synthesized, including proteins, from that target RNA. Oligonucleotide inhibitors having an antiviral activity that is based on hybridizing to a complementary sequence are distinguished from oligonucleotides having sequence independent antiviral activity on the basis of both their structure (length) and function (mechanism of action).

As used herein, “a” or “an” may mean one or more than one.

As used herein, the term “about” has its usual meaning as understood by those skilled in the art and thus indicates that a value includes the inherent variation of error for the method being employed to determine a value, or the variation that exists among multiple determinations.

The terms “individual”, “subject”, or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, and the like.

The terms “effective amount” or “effective dose” as used herein have their usual meaning as understood by those skilled in the art, and refer to that amount of a recited composition or compound that results in an observable biological effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

As used herein, the term “pharmaceutically acceptable” has its usual meaning as understood by those skilled in the art and refers to carriers, diluents, excipients, and/or stabilizers that are nontoxic to the cell or mammal being exposed thereto at the dosages and concentrations employed or that have an acceptable level of toxicity. The terms “diluent,” “excipient,” and/or “carrier” as used herein have their usual meaning as understood by those skilled in the art and are include any and all solvents, dispersion media, coatings, antibacterial and antifungal agents, isotonic and absorption delaying agents, and the like, compatible with administration to humans, mice, rats, cats, dogs, or other vertebrate hosts.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

### Hepatitis B, D, and Other Liver Diseases

The hepatitis B virus (HBV) is a DNA virus and a member of the Hepadnaviridae family. HBV infects more than 300 million worldwide and is a causative agent of liver cancer and liver disease such as chronic hepatitis, cirrhosis, and hepatocellular carcinoma. HBV can be acute and/or chronic. Acute HBV infection can be either asymptomatic or present with symptomatic acute hepatitis. HBV is classified into eight genotypes, A to H.

HBV is a partially double-stranded circular DNA of about 3.2 kilobase (kb) pairs. The HBV replication pathway has been studied in great detail. T. J. Liang, Heptaology (2009) 49(5 Suppl):S13-S21. One part of replication includes the formation of the covalently closed circular (cccDNA) form. The presence of the cccDNA gives rise to the risk of viral reemergence throughout the life of the host organism. HBV carriers can transmit the disease for many years. An estimated 257 million people are living with hepatitis B virus infection, and it is estimated that over 750,000 people worldwide die of hepatitis B each year. In addition, immunosuppressed individuals or individuals undergoing chemotherapy are especially at risk for reactivation of an HBV infection.

HBV can be transmitted by blood, semen, and/or another body fluid. This can occur through direct blood-to-blood contact, unprotected sex, sharing of needles, and from an infected mother to her baby during the delivery process. The HBV surface antigen (HBsAg) is most frequently used to screen for the presence of this infection. Currently available medications do not cure an HBV and/or HDV infection. Rather, the medications suppress replication of the virus.

The hepatitis D virus (HDV) is a DNA virus, also in the Hepadnaviridae family of viruses. HDV can propagate only in the presence of HBV. The routes of transmission of HDV are similar to those for HBV. Transmission of HDV can occur either via simultaneous infection with HBV (coinfection) or in addition to chronic hepatitis B or hepatitis B carrier state (superinfection). Both superinfection and coinfection with HDV results in more severe complications compared to infection with HBV alone. These complications include a greater likelihood of experiencing liver failure in acute infections and a rapid progression to liver cirrhosis, with an increased risk of developing liver cancer in chronic infections. In combination with hepatitis B, hepatitis D has the highest fatality rate of all the hepatitis infections, at 20%. There is currently no cure or vaccine for hepatitis D.

### Oligonucleotide Conjugates and Complexes Thereof

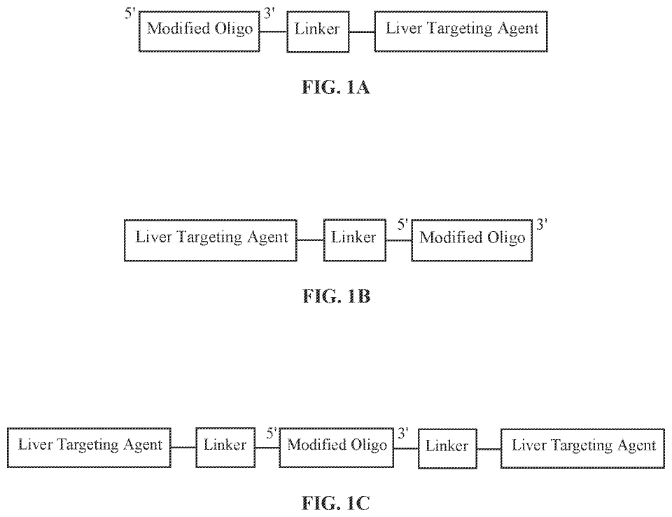

Various embodiments provide an oligonucleotide conjugate or complex thereof that comprises a modified oligonucleotide having sequence independent antiviral activity against HBV, a liver targeting agent and a linker connecting the liver targeting agent to a 3′ end and/or a 5′ end of the modified oligonucleotide or complex thereof. FIGS. 1A, 1B and 1C schematically illustrate non-limiting examples of such oligonucleotide conjugates or complexes thereof.

Various modified oligonucleotides (and complexes thereof) having sequence independent antiviral activity against HBV are known to those skilled in the art and may be incorporated into the oligonucleotide conjugates and complexes thereof as described herein. In an embodiment, the modified oligonucleotide or complex thereof having sequence independent antiviral activity against HBV is partially or fully phosphorothioated. For example, in an embodiment, the modified oligonucleotide or complex thereof is an at least partially phosphorothioated modified oligonucleotide or complex thereof (such as a STOPS™ antiviral compound) as described in WO 2020/097342, WO 2021/119325, US 2020/0147124 and/or U.S. Pat. No. 11,166,976, each of which are hereby incorporated herein by reference and particularly for the purpose of describing at least partially phosphorothioated modified oligonucleotides and complexes thereof having sequence independent antiviral activity against HBV that is not based on hybridizing to a complementary sequence. In another embodiment, the modified oligonucleotide or complex thereof having sequence independent antiviral activity against HBV is a phosphorothioated oligonucleotide or complex thereof as described in U.S. Pat. No. 9,603,865 (such as REP 2055, REP 2139, REP 2163 or REP 2165), which is hereby incorporated herein by reference and particularly for the purpose of describing phosphorothioated oligonucleotides and complexes thereof having sequence independent antiviral activity against HBV.

Specific examples of modified oligonucleotides having sequence independent antiviral activity against HBV are described in Table 1.

As disclosed herein, the inventors discovered that the structure of the conjugate unexpectedly impacts the targeting of the conjugate to the liver. Specific examples of these findings are described in the working examples below.

TABLE 1
MODIFIED OLIGONUCLEOTIDES

| Oligo No. | Sequence (5′→3′) |
|---|---|
| Oligo 1 | lnApsln(5m)CpslnApsln(5m)CpslnApsln |
| (SEQ ID | (5m)CpslnApsln(5m)CpslnApsln(5m) |
| NO: 1) | CpslnApsln(5m)CpslnApsln(5m) |
| | CpslnApsln(5m)CpslnApsln(5m) |
| | CpslnApsln(5m)CpslnApsln(5m) |
| | CpslnApsln(5m)CpslnApsln(5m) |
| | CpslnApsln(5m)CpslnApsln(5m) |
| | CpslnApsln(5m)CpslnApsln(5m) |
| | CpslnApsln(5m)CpslnApsln(5m) |
| | CpslnApsln(5m)C |
| Oligo 2 | mApsm(5m)CpsmApsm(5m)CpsmApsm(5m) |
| (REP 2139) | CpsmApsm(5m)CpsmApsm(5m)CpsmApsm |
| (SEQ ID | (5m)CpsmApsm(5m)CpsmApsm(5m)CpsmApsm |
| NO: 2) | (5m)CpsmApsm(5m)CpsmApsm(5m)CpsmApsm |
| | (5m)CpsmApsm(5m)CpsmApsm(5m)CpsmApsm |
| | (5m)CpsmApsm(5m)CpsmApsm(5m)CpsmApsm |
| | (5m)CpsmApsm(5m)CpsmApsm(5m)C |
| Oligo 3 | mApsln(5m)CpsmApsln(5m)CpsmApsln(5m) |
| (SEQ ID | CpsmApsln(5m)CpsmApsln(5m)CpsrApsln |
| NO: 3) | (5m)CpsmApsln(5m)CpsmApsln(5m) |
| | CpsrApsln(5m)CpsmApsln(5m)CpsmApsln |
| | (5m)CpsrApsln(5m)CpsmApsln(5m)CpsmApsln |
| | (5m)CpsrApsln(5m)CpsmApsln(5m) |
| | CpsmApsln(5m)CpsrApsln(5m)CpsmApsln |
| | (5m)CpsmApsln(5m)C |
| Oligo 4 | mApsln(5m)CpsmApsln(5m)CpsmApsln(5m) |
| (SEQ ID | CpsmApsln(5m)CpsmApsln(5m)CpsrApsln |
| NO: 4) | (5m)CpsmApsln(5m)CpsmApsln(5m) |
| | CpsrApsln(5m)CpsmApsln(5m)CpsmApsln |
| | (5m)CpsrApsln(5m)CpsmApsln(5m)CpsmApsln |
| | (5m)CpsrApsln(5m)CpsmApsln(5m) |
| | CpsmApsln(5m)CpsmApsln(5m)CpsmApsln |
| | (5m)CpsmApsln(5m)C |
| Oligo 5 | mApsln(5m)CpsmApsBpsmApsln(5m) |
| (SEQ ID | CpsmApsln(5m)CpsmApsBpsmApsln(5m) |
| NO: 5) | CpsmApsln(5m)CpsmApsBpsmApsln(5m) |
| | CpsmApsln(5m)CpsmApsBpsmApsln(5m) |
| | CpsmApsln(5m)CpsmApsBpsmApsln(5m) |
| | CpsmApsln(5m)CpsmApsBpsmApsln(5m) |
| | CpsmApsln(5m)CpsmApsln(5m)C |
| Oligo 6 | mApsm(5m)CpsmApsm(5m)CpsmApsm(5m) |
| (REP-2165) | CpsmApsm(5m)CpsmApsm(5m)CpsrApsm(5m) |
| (SEQ ID | CpsmApsm(5m)CpsmApsm(5m)CpsmApsm(5m) |
| NO: 6) | CpsmApsm(5m)CpsrApsm(5m)CpsmApsm(5m) |
| | CpsmApsm(5m)CpsmApsm(5m)CpsmApsm(5m) |
| | CpsrApsm(5m)CpsmApsm(5m)CpsmApsm(5m) |
| | CpsmApsm(5m)CpsmApsm(5m)C |
| Oligo 7 | lnApsm(5m)CpsmApsm(5m)CpsmApsm(5m) |
| (SEQ ID | CpsmApsm(5m)CpsmApsm(5m)CpsrApsm(5m) |
| NO: 7) | CpsmApsm(5m)CpsmApsm(5m)CpsmApsm(5m) |
| | CpsmApsm(5m)CpsrApsm(5m)CpsmApsm(5m) |
| | CpsmApsm(5m)CpsmApsm(5m)CpsmApsm(5m) |
| | CpsrApsm(5m)CpsmApsm(5m)CpsmApsm(5m) |
| | CpsmApsm(5m)CpsmApsln(5m)C |

Abbreviations used in Table 1 and elsewhere in this disclosure are summarized in Table 2.

TABLE 2
ABBREVIATIONS

| Abbreviation | Name or Structure |
|---|---|
| A | Deoxy-Adenosine |
| A-A-A | Same as “po-A-po-A-po-A-po” |
| lnA | 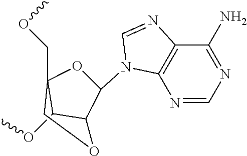 |
| mA | 2′-O-Methyladenosine |
| rA | Ribo-Adenosine, Adenosine |
| B | |
| C | Cytidine |
| C-C-C | Same as “po-C-po-C-po-C-po” |
| ln(5m)C | 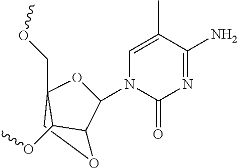 |
| m(5m)C | 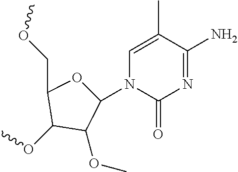 |
| (5m)C | 5-methylcytidine |
| C₆ | Hexyl |
| HEG | Hexaethylene glycol |
| HEG-HEG | Same as “po-HEG-po-HEG-po” |
| po (or p) | Phosphodiester |
| ps | PO₃S (Phosphorothioate) |
| GalNAc1 | |
| GalNac2 | 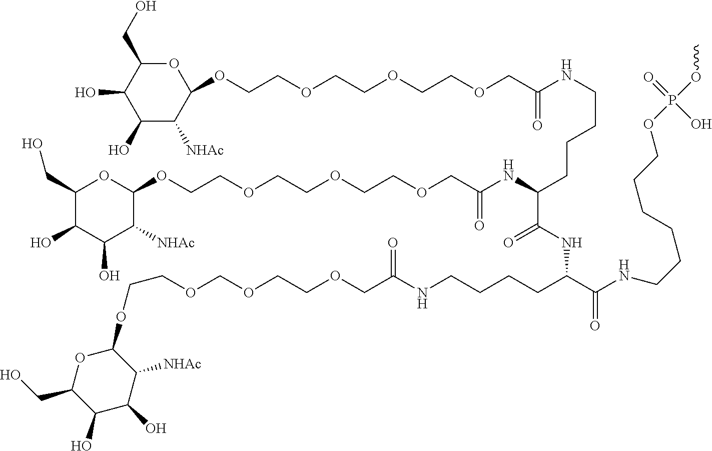 |
| GalNac3 | |
| GalNac4 | |
| GalNac5 | 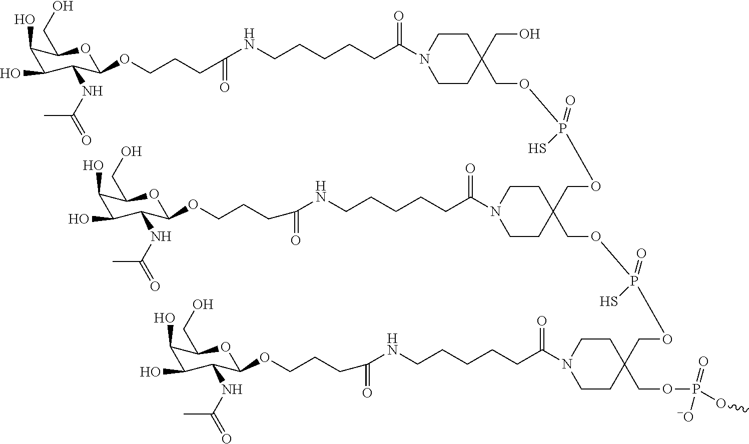 |
| GalNac6 | 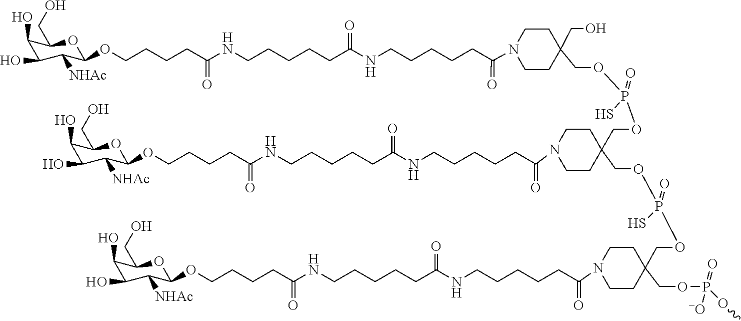 |
| Tocopherol | 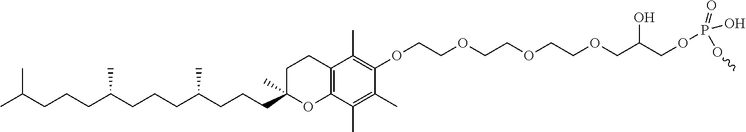 |
| Cholesterol | 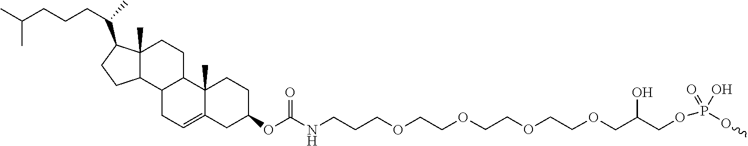 |
| Palmitoyl | 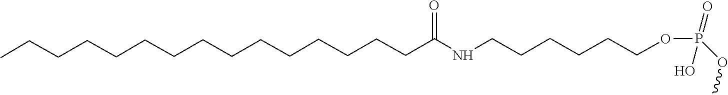 |

Various liver targeting agents may be incorporated into the oligonucleotide conjugates and complexes thereof as described herein and illustrated in FIGS. 1A-C. As disclosed herein, one or more liver targeting agents may be connected to the 5′, 3′, or both ends of the modified oligonucleotide. The linkage of the liver targeting agent(s) to the modified oligonucleotide may be direct (via covalent bond) or indirect (via a linking group). Various liver targeting agents that enhance delivery of a connected moiety to the liver may be used. In various embodiments, the liver targeting agent is GalNac1, GalNac2, GalNac3, GalNac4, GalNac5, GalNac6, tocopherol, cholesterol, a palmitoyl/palmitate group, a C₁₂₋₂₀ alkyl ester or a C₁₂₋₂₀ alkenyl ester.

Various linkers may be incorporated into the oligonucleotide conjugates and complexes thereof as described herein and illustrated in FIGS. 1A-C. The linker(s) connects the modified oligonucleotide to the liver targeting agent(s). Those skilled in the art will recognize that such connection may include phosphodiester or phosphorothioate as appropriate. Various types of linkers may be used, such as a covalent bond, a cleavable linker, or a non-cleavable linker.

In various embodiments the linker is a covalent bond. In other embodiments, improved liver targeting results at least in part from including a linker that provides increased spacing between the modified oligonucleotide and the liver targeting agent(s) as compared to a covalent bond. In such embodiments, the linker may be referred to herein as a spacer group or simply a spacer. In an embodiment, the linker is a C₄₋₈ alkyl spacer, such as butyl, pentyl, hexyl, heptyl or octyl. In other embodiments the linker comprises a polyethylene glycol (PEG) spacer, optionally with phosphodiester (po). For example, in an embodiment, the linker is -po-O-(CH₂CH₂O)ₘ-po- or -po[-O-(CH₂CH₂O)ₘ-po-O-(CH₂CH₂O)ₙ]-po, wherein m and n are each independently 2, 3, 4, 5, 6, 7, 8, 9 or 10. In an embodiment, the linker comprises a Hexaethylene glycol (HEG) spacer, e.g., -po-HEG-po- or -po-HEG-po-HEG-po-.

In some embodiments, the linker is a spacer that comprises or consists of one or more nucleotides. Linkers comprising multiple nucleotides may subsist of the same nucleotide, or any combination of nucleotides. The one or more nucleotide can be but is not limited to adenosine, cytidine, guanosine, thymidine, uridine, or any combination thereof. Unless otherwise specified, multiple nucleotides are connected by a phosphodiesters or phosphorothioate in between each nucleotide. This is denoted as either po (phosphodiester) or ps (phosphorothioate). If there is no annotation, the connection is via a phosphodiester. The one or more nucleotide can be in its ribose form (r), its deoxy form, or any chemically modified form, such as but not limited to bridged/locked (ln), methylated (m), di-methylated m(m), tri-methylated, or any combination thereof. For example, the linker can be A, A-A, A-A-A, A-A-A-A, A-A-A-A-A, mA, mA-mA, mA-mA-mA, mA-mA-mA-mA, mA-mA-mA-mA-mA, rA, rA-rA, rA-rA-rA, rA-rA-rA-rA, rA-rA-rA-rA-rA, A-C, C-A, A-C-A-C, C-A-C-A, A-C-A-C-A-C, C-A-C-A-C-A, A-C-A-C-A-C-A-C, C-A-C-A-C-A-C-A, A-C-A-C-A-C-A-C-A-C, C-A-C-A-C-A-C-A-C-A or mA-lnC-mA-lnC. In some embodiments, the methylated adenosine is 2′-O-methyladenosine. In some embodiments, the methylated cytidine is 2′-O-methylcytidine. In some embodiments, the methylated cytidine is 5-methylcytidine. In some embodiments, the di-methylated cytidine is 2′-O-methyl-5-methylcytidine. In some embodiments, the linker is -po-(Nuc-po)ₓ-, wherein each Nuc nucleotide in -po-(Nuc-po)ₓ- is independently an A nucleotide or a C nucleotide; each A nucleotide in -po-(Nuc-po)ₓ- is independently adenosine, 2′-O-methyladenosine, or deoxyadenosine, or any combination thereof; each C nucleotide in -po-(Nuc-po)ₓ- is independently cytidine, 2′-O-methylcytidine, 5-methylcytidine, 2′-O-5-dimethylcytidine, a locked/bridged methylcytidine, ribose cytidine, or deoxycytidine, or any combination thereof; po is phosphodiester; and each x is independently an integer 1, 2, 3, 4 or 5.

In an embodiment, at least one nucleotide in -po-(Nuc-po)ₓ- is adenosine. In another embodiment, at least one nucleotide in -po-(Nuc-po)ₓ- is cytidine. In an embodiment, at least one nucleotide in -po-(Nuc-po)ₓ- is a deoxy-nucleotide. In another embodiment, at least one nucleotide in -po-(Nuc-po)ₓ- is a 2′-O-methylated nucleotide. In another embodiment, at least one nucleotide in -po-(Nuc-po)ₓ- is a ribo-nucleotide. In another embodiment, at least one C nucleotide in -po-(Nuc-po)ₓ- is 5-methylcytidine. In another embodiment, at least one C nucleotide in -po-(Nuc-po)ₓ- is 2′-O-methyl-5-methylcytidine. In another embodiment, at least one C nucleotide in -po-(Nuc-po)ₓ- is a locked/bridged cytidine or a locked/bridged methylcytidine.

The linker described herein may be selected on the basis of its length when used as a spacer to control the overall distance from the modified oligonucleotide to the liver targeting agent it connects. Further, the linker described herein may be selected on the basis of its influence on the physicochemical properties, solubility properties, and/or other properties of the oligonucleotide conjugate and/or modified oligonucleotide with which it is included. As disclosed herein, the inclusion of a linker that provides spacing enhances targeting of the modified oligonucleotide to the liver of a subject (e.g., mouse, non-human primate, or human) to which the oligonucleotide conjugate or complex thereof is administered. Such targeting enhancement may take the form of an increase in the concentration of the modified oligonucleotide that reaches the liver, an increase in the ratio of the amount of the modified oligonucleotide reaching the liver compared with another organ (such as kidney), or both, as compared to the oligonucleotide conjugate without the linker.

Unexpectedly, the inventors found that the properties of a linker in enhancing the concentration of modified oligonucleotide in the liver and/or the ratio of modified oligonucleotide in the liver compared with other organs (such as kidney) is not solely dependent on the length of the linker. Rather, a combination of the length of the linker, the chemical properties of the linker and the chemical properties of the liver targeting agent play a large role in liver targeting. For example, in an embodiment, the liver targeting agent and the linker are selected to provide improved targeting of the modified oligonucleotide to the liver of a mouse, non-human primate, human, or other mammalian subject to which the oligonucleotide conjugate or complex thereof is administered, wherein the improved targeting is determined by comparison to a corresponding unconjugated modified oligonucleotide at a 6-hour, 24-hour, or 48-hour timepoint following subcutaneous administration to a mouse, primate, human, or other mammalian subject at a dosage of about 0.05, 0.1, 0.15, 0.25, 0.5, 0.75, 1, 2, 3, 4, 5, or 15 mg/kg. The unconjugated modified oligonucleotide lacks both the linker and the liver targeting agent and thus the comparison illustrates the combined impact of including them in the oligonucleotide conjugate or complex thereof.

In an embodiment, the linker is a spacer that provides increased spacing between the modified oligonucleotide and the liver targeting agent(s) as compared to a covalent bond. In an embodiment, the liver targeting agent and the spacer are selected to provide improved targeting of the modified oligonucleotide to the liver of a mouse, non-human primate, human, or other mammalian subject to which the oligonucleotide conjugate or complex thereof is administered, wherein the improved targeting is determined by comparison to a corresponding modified oligonucleotide conjugate that lacks the spacer at a 6-hour, 24-hour, or 48-hour timepoint following subcutaneous administration to a mouse, primate, human, or other mammalian subject at a dosage of about 0.05, 0.1, 0.15, 0.25, 0.5, 0.75, 1, 2, 3, 4, 5, or 15 mg/kg. In such an embodiment, the linker is a spacer and the comparison is to a corresponding oligonucleotide conjugate lacking the spacer but having the same liver targeting agent, such that the comparison illustrates the impact of including the spacer. In various embodiments, the linker and targeting agent together form a ligand of the general formula -po-(Nuc-po)ₓ-(GalNacX), wherein x is 1, 2 or 3 and X is 1, 2, 3, 4, 5 or 6. In some embodiments, Nuc is A, x is 1, 2 or 3 and X is 4, as exemplified by -po-A-po-GalNac4, -po-A-po-A-po-GalNac4 and -po-A-po-A-po-A-po-GalNac4. In various embodiments, such ligands can be attached to the oligonucleotide at the 5′ end, the 3′ end, or both.

Improved liver targeting can be determined directly by evaluating the accumulation of the modified oligonucleotide in a mammalian model system such as a single liver cell, liver cell culture, liver tissue, liver organ, liver organoid, or whole organism from a mouse, non-human primate, human, or other mammal. Improved liver targeting may also be determined indirectly by evaluating the accumulation of the modified oligonucleotide in a different organ or organ model system, to the extent that reduced accumulation in that organ is correlated with increased liver targeting, on an absolute and/or relative basis (e.g., as indicated by an increase in the ratio of the concentration in the liver to the concentration in the other organ). In some embodiments, the oligonucleotide conjugate is administered parenterally, e.g., intravenously or subcutaneously. In some embodiments, the oligonucleotide conjugate is administered (e.g., to a human, primate, or mouse) at a dosage of about 0.05, 0.1, 0.15, 0.25, 0.5, 0.75, 1, 2, 3, 4, 5, 6, 7, 8, 9, 10, 11, 12, 13, 14 or 15 mg/kg. The concentration of the resultant modified oligonucleotide is then measured in the CD-1 mouse liver model (Charles River) at a timepoint that is about 6, about 8, about 10, about 12, about 14, about 16, about 18, about 20, about 22, about 24, about 26, about 28, about 30, about 32, about 34, about 36, about 38, about 40, about 42, about 44, about 46, or about 48 hours after administration.

Liver targeting can be evaluated directly by determining the concentration of the modified oligonucleotide in the liver. In some embodiments, the linker and liver targeting agent are selected to provide improved targeting of the modified oligonucleotide to the liver as evidenced by a liver concentration that is at least about 2,000 ng/g, at least about 8,000 ng/g, at least about 9,000 ng/g, at least about 10,000 ng/g, at least about 11,000 ng/g, at least about 12,000 ng/g, at least about 13,000 ng/g, at least about 18,000 ng/g, at least about 40,000 ng/g, at least about 50,000 ng/g, at least about 60,000 ng/g, or in a range defined by any two of the foregoing liver concentration values as endpoints. Liver targeting can be evaluated indirectly by determining the concentration of the modified oligonucleotide in the kidney. In some embodiments, the linker and liver targeting agent are selected to provide improved targeting of the modified oligonucleotide to the liver as evidenced by a kidney concentration that is less than about 10,000 ng/g, less than about 9,000 ng/g, less than about 8,000 ng/g, less than about 7,000 ng/g, less than about 6,000 ng/g, less than about 5,000 ng/g, or less than about 4,000 ng/g. Liver targeting can be also evaluated indirectly by determining the ratio of the concentration of modified oligonucleotide in the liver to the concentration in another organ type, such as the kidney. In some embodiments, the linker and liver targeting agent are selected to provide improved targeting of the modified oligonucleotide to the liver as evidenced by a ratio of the concentration in the liver to the concentration in the kidney that is at least about 0.8, at least about 0.9, at least about 1.0, at least about 1.1, at least about 1.2, at least about 1.3, at least about 1.4, at least about 1.5, at least about 1.6, at least about 1.7, at least about 1.8, at least about 1.9, at least about 2.0, at least about 2.1, at least about 2.2, at least about 2.3, at least about 2.4, at least about 2.5, at least about 2.6, at least about 2.7, at least about 2.8, at least about 2.9, or at least about 3.0. Specific examples of the impact of including a linker (covalent bond or spacer) and a targeting agent, as well as the differences in effectiveness between types of linkers and targeting agents, are described in the Examples below.

### Pharmaceutical Compositions

The oligonucleotide conjugates and complexes thereof described herein may be incorporated into a pharmaceutical composition for treating a hepatitis B infection by administration to a subject in need thereof. In some embodiments, the subject is a human. In some embodiments, the subject is a non-human primate model. In some embodiments, the subject is a mouse model. In some embodiments, the subject has a liver disease or disorder that results from a hepatitis B viral infection.

Some embodiments described herein relate to pharmaceutical compositions that comprise, consist essentially of, or consist of an effective amount of an oligonucleotide conjugate composition described herein and a pharmaceutically acceptable carrier, diluent, excipient, or combination thereof. A pharmaceutical composition described herein is suitable for human and/or veterinary applications.

Various pharmaceutically acceptable carriers, diluents, excipients, or combinations thereof can be incorporated into a pharmaceutical composition. In an embodiment, the pharmaceutically acceptable diluent, excipient, and/or carrier is one that is approved by a government regulatory agency, or listed in the U.S. Pharmacopeia or other generally recognized pharmacopeia for use in animals, including humans as well as non-human mammals, such as cats, dogs, non-human primates, and mice. The pharmaceutically acceptable diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a pharmaceutically acceptable carrier is an aqueous pH buffered solution. The pharmaceutically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, nonionic surfactants such as TWEEN®, polyethylene glycol (PEG), HEG, PLURONICS®, nanoparticles, nanosomes, micelles, lipid nanoparticles, and liposomes. Suitable pharmaceutical carriers also include any molecule or solvent that impacts the delivery, uptake, and metabolism of a drug, protein, or molecule. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

### Uses and Methods of Treatment

Various embodiments provide a method of treating a liver disease or disorder, comprising administering an effective amount of an oligonucleotide conjugate or complex thereof as described herein to a subject in need thereof. In an embodiment, the oligonucleotide conjugate or complex thereof is administered to the subject in the form of a pharmaceutical composition as described herein. In some embodiments, the liver disease or disorder is an HBV infection, which can be an acute HBV infection or a chronic HBV infection. In some embodiments, the liver disease or disorder is a liver cirrhosis that is developed because of an HBV infection. In an embodiment, such a method of treating liver cirrhosis that is developed because of a HBV infection comprises subcutaneous administration of the oligonucleotide conjugate or complex thereof to a human at a dosage lower than otherwise expected based on liver levels observed following otherwise comparable intravenous administration. In some embodiments, the oligonucleotide conjugate or complex thereof comprises a modified oligonucleotide that is a highly potent STOPS™ antiviral compound as described in WO 2020/097342, WO 2021/119325, US 2020/0147124 and/or U.S. Pat. No. 11,166,976. In an embodiment, the oligonucleotide conjugate is described in Table 3. In various embodiments, the effective amount of oligonucleotide conjugate that is administered to the subject (e.g., to a human, non-human primate, or mouse) is a dosage of about 0.05 mg/kg, about 0.06 mg/kg, about 0.07 mg/kg, about 0.08 mg/kg, about 0.09 mg/kg, about 0.1 mg/kg, about 0.25 mg/kg, about 0.5 mg/kg, about 0.75 mg/kg, about 1 mg/kg, about 5 mg/kg, about 10 mg/kg, about 15 mg/kg, or in a dosage range defined by any two of the foregoing dosage values as endpoints.

Various routes may be used to administer an oligonucleotide conjugate or complex thereof to a subject in need thereof. In an embodiment, an effective amount of the oligonucleotide conjugate or complex thereof, or of a pharmaceutical composition that includes such an oligonucleotide conjugate or complex thereof, is administered to the subject by a parenteral route. For example, in an embodiment, an effective amount of the oligonucleotide conjugate or complex thereof, or of a pharmaceutical composition that includes the oligonucleotide conjugate or complex thereof, is administered to the subject intravenously. In another embodiment, an effective amount of the oligonucleotide conjugate or complex thereof, or of a pharmaceutical composition that includes such an oligonucleotide conjugate or complex thereof, is administered to the subject subcutaneously.

Some embodiments described herein relate to a method of treating a liver disease or disorder as described herein (e.g., HBV infection or a liver cirrhosis that is developed because of a HBV infection) that can include administering, to a subject identified as suffering from the liver disease or disorder, an effective amount of an oligonucleotide conjugate or complex thereof as described herein, or a pharmaceutical composition that includes such an oligonucleotide conjugate or complex thereof. Other embodiments described herein relate to using an oligonucleotide conjugate or complex thereof as described herein, or a pharmaceutical composition that includes such an oligonucleotide conjugate or complex thereof, in the manufacture of a medicament for treating a liver disease or disorder as described herein. Still other embodiments relate to the use of an oligonucleotide conjugate or complex thereof as described herein, or a pharmaceutical composition that includes such an oligonucleotide conjugate or complex thereof, for treating a liver disease or disorder as described herein.

Various embodiments provide methods of treating a liver disease or disorder (e.g., HBV infection or a liver cirrhosis that is developed because of a HBV infection) by administering an effective amount of an oligonucleotide conjugate or complex thereof to a subject as described herein. Other embodiments provide oligonucleotide conjugates or complexes thereof as described herein for use in the treatment of such liver diseases or disorders and/or for use in the manufacture of medicaments for such treatments. In various embodiments, such methods and uses further comprise administering an effective amount of at least one second therapy to the subject in combination with the oligonucleotide conjugate or complex thereof. The at least one second therapy may be selected from various therapeutic modalities for treatment of the liver disease or disorder. In an embodiment, the at least one second therapy comprises administering an effective amount of at least one second therapeutic molecule to the subject in combination with the oligonucleotide conjugate or complex thereof. For example, in various embodiments, the at least one second therapeutic molecule comprises a second oligonucleotide conjugate or complex thereof as described herein, a nucleoside, a nucleotide, a nucleotide prodrug, an interferon, a capsid assembly modulator, a protease inhibitor, or a combination thereof.

### EXAMPLES

Additional embodiments are disclosed in further detail in the following examples, which are not in any way intended to limit the scope of the claims.

### Examples 1-50

Oligonucleotide conjugates 1-50, comprising various modified oligonucleotides, linkers and liver targeting agents were prepared as summarized in Table 3. The conjugated oligonucleotides were synthesized using standard phosphoramidite chemistry and characterized by LC-MS for integrity and HPLC for purity.

TABLE 3

| Conjugate No. | 5' end | Linker | 3' end |
|---|---|---|---|
| Conjugate 1 | Oligo 4 | Covalent bond | Ga1Nac4 |
| (SEQ. ID NO:8) | | | |
| Conjugate 2 | Cholesterol | Covalent bond | Oligo 4 |
| (SEQ. ID NO:9) | | | |
| Conjugate 3 | GalNac4 | Covalent bond | Oligo 4 |
| (SEQ. ID NO:10) | | | |
| Conjugate 4 | GalNac6 | Covalent bond | Oligo 4 |
| (SEQ. ID NO:11) | | | |
| Conjugate 5 | GalNac4- | Covalent bond | GalNac4 |
| (SEQ. ID NO:12) | Oligo 4 | | |
| Conjugate 6 | Cholesterol | poHEGpo | Oligo 4 |
| (SEQ. ID NO:13) | | | |
| Conjugate 7 | GalNac2 | Hexyl | Oligo 4 |
| (SEQ. ID NO:14) | | | |
| Conjugate 8 | GalNac4 | poApo | Oligo 4 |
| (SEQ. ID NO:15) | | | |
| Conjugate 9 | GalNac4 | poApoApo | Oligo 4 |
| (SEQ. ID NO:16) | | | |
| Conjugate 10 | GalNac4 | poApoApoApo | Oligo 2 |
| (SEQ. ID NO:17) | | | |
| Conjugate 11 | GalNac4 | poApoApoApo | Oligo 4 |
| (SEQ. ID NO:18) | | | |
| Conjugate 12 | GalNac4 | poHEGpo | Oligo 4 |
| (SEQ. ID NO:19) | | | |
| Conjugate 13 | GalNac4 | poHEGpoHEGpo | Oligo 4 |
| (SEQ. ID NO:20) | | | |
| Conjugate 14 | Ga1Nac4 | pomApoln(5m)Cpom | Oligo 4 |
| (SEQ. ID NO: 21) | | Apoln(5m)Cpo | |
| Conjugate 15 | Ga1Nac4 | pomApomApomApo | Oligo 4 |
| (SEQ. ID NO: 22) | | | |
| Conjugate 16 | Ga1Nac4 | porAporAporApo | Oligo 4 |
| (SEQ. ID NO: 23) | | | |
| Conjugate 17 | GalNac6 | poApoApoApo | Oligo 4 |
| (SEQ. ID NO: 24) | | | |
| Conjugate 18 | Palmitoyl | poApoApoApo | Oligo 4 |
| (SEQ. ID NO: 25) | | | |
| Conjugate 19 | Palmitoyl | poHEGpo | Oligo 4 |
| (SEQ. ID NO: 26) | | | |
| Conjugate 20 | Oligo 1 | Covalent bond | GalNac2 |
| (SEQ. ID NO: 27) | | | |
| Conjugate 21 | GalNac2 | Covalent bond | Oligo 1 |
| (SEQ. ID NO: 28) | | | |
| Conjugate 22 | GalNac4 | Covalent bond | Oligo 1 |
| (SEQ. ID NO: 29) | | | |
| Conjugate 23 | Oligo 1 | poCpoApo | GalNac2 |
| (SEQ. ID NO: 30) | | | |
| Conjugate 24 | GalNac2 | poCpoApo | Oligo 1 |
| (SEQ. ID NO: 31) | | | |
| Conjugate 25 | GalNac4 | poApo | Oligo 3 |
| (SEQ. ID NO: 32) | | | |
| Conjugate 26 | GalNac4 | poApoApo | Oligo 3 |
| (SEQ. ID NO: 33) | | | |
| Conjugate 27 | GalNac4 | poApoApoApo | Oligo 3 |
| (SEQ. ID NO: 34) | | | |
| Conjugate 28 | GalNac6 | poApo | Oligo 3 |
| (SEQ. ID NO: 35) | | | |
| Conjugate 29 | GalNac6 | poApoApo | Oligo 3 |
| (SEQ. ID NO: 36) | | | |
| Conjugate 30 | GalNac6 | poApoApoApo | Oligo 3 |
| (SEQ. ID NO: 37) | | | |
| Conjugate 31 | GalNac4 | porApo | Oligo 3 |
| (SEQ. ID NO: 38) | | | |
| Conjugate 32 | GalNac4 | porAporApo | Oligo 3 |
| (SEQ. ID NO: 39) | | | |
| Conjugate No. | 5' end | Linker | 3' end |
| Conjugate 33 | Ga1Nac4 | porAporAporApo | Oligo 3 |
| (SEQ. ID NO: 40) | | | |
| Conjugate 34 | Ga1Nac6 | porApo | Oligo 3 |
| (SEQ. ID NO: 41) | | | |
| Conjugate 35 | Ga1Nac6 | porAporApo | Oligo 3 |
| (SEQ. ID NO: 42) | | | |
| Conjugate 36 | Ga1Nac6 | porAporAporApo | Oligo 3 |
| (SEQ. ID NO: 43) | | | |
| Conjugate 37 | Ga1Nac4 | pomApo | Oligo 3 |
| (SEQ. ID NO: 44) | | | |
| Conjugate 38 | Ga1Nac4 | pomApomApo | Oligo 3 |
| (SEQ. ID NO: 45) | | | |
| Conjugate 39 | Ga1Nac4 | pomApomApomApo | Oligo 3 |
| (SEQ. ID NO: 46) | | | |
| Conjugate 40 | Ga1Nac6 | pomApo | Oligo 3 |
| (SEQ. ID NO: 47) | | | |
| Conjugate 41 | Ga1Nac6 | pomApomApo | Oligo 3 |
| (SEQ. ID NO: 48) | | | |
| Conjugate 42 | Ga1Nac6 | pomApomApomApo | Oligo 3 |
| (SEQ. ID NO: 49) | | | |
| Conjugate 43 | Ga1Nac4 | poHEGpo | Oligo 3 |
| (SEQ. ID NO: 50) | | | |
| Conjugate 44 | GalNac4 | poHEGpoHEGpo | Oligo 3 |
| (SEQ. ID NO: 51) | | | |
| Conjugate 45 | GalNac6 | poHEGpo | Oligo 3 |
| (SEQ. ID NO: 52) | | | |
| Conjugate 46 | GalNac6 | poHEGpoHEGpo | Oligo 3 |
| (SEQ. ID NO: 53) | | | |
| Conjugate 47 | GalNac4 | Covalent bond | Oligo 6 |
| (SEQ. ID NO: 54) | | | |
| Conjugate 48 | GalNac4 | poApoApoApo | Oligo 6 |
| (SEQ. ID NO: 55) | | | |
| Conjugate 49 | GalNac4 | Covalent bond | Oligo 7 |
| (SEQ. ID NO: 56) | | | |
| Conjugate 50 | GalNac4 | poApoApoApo | Oligo 7 |
| (SEQ. ID NO: 57) | | | |

### Examples A1-A4

Modified oligonucleotides 1, and 3-5 (unconjugated), were administered subcutaneously to the interscapular region of CD-1 mice (Charles River, n=4) at a dosage of 15 mg/kg (Oligo 1, SEQ ID NO: 1) or 5 mg/kg (Oligo 3, Oligo 4 and Oligo 5; SEQ ID NOS: 3, 4, and 5, respectively) using standard methods. The resulting concentrations of the modified oligonucleotides in the livers of the test subjects at the 24 hour timepoint after administration were determined using standard LC-MS/MS methods, as summarized in Tables 4A and 4B.

TABLE 4A

| | Oligo Conc. in |
|---|---|
| Oligo No. (15 mg/kg) | Liver (ng/g) |
| Oligo 1 | 9,187 |

TABLE 4B

| Oligo No. | Oligo Conc. in |
|---|---|
| (5 mg/kg) | Liver (ng/g) |
| Oligo 3 | 4,571 |
| (SEQ. ID NO: 3) | |
| Oligo 4 | 4,237 |
| (SEQ. ID NO: 4) | |
| Oligo 5 | 12,125 |
| (SEQ. ID NO: 5) | |

### Examples B1-B19

Oligonucleotide conjugates were administered subcutaneously to the interscapular region of CD-1 mice (Charles River, n=4) at a dosage of 5 mg/kg using standard methods. The resulting concentrations of the modified oligonucleotides in the livers of the test subjects at the 24 hour timepoint after administration were determined using standard LC-MS/MS methods, as summarized in Table 5.

TABLE 5

| | | | | Oligo |
|---|---|---|---|---|
| Conjugate No. | 5' end | Linker | 3' end | Conc.In Liver (ng/g) |
| Conjugate 1 | Oligo 4 | Covalent bond | GalNac4 | Not detected |
| (SEQ. ID NO: 8) | | | | |
| Conjugate 2 | Cholesterol | Covalent bond | Oligo 4 | 6,448 |
| (SEQ. ID NO: 9) | | | | |
| Conjugate 3 | GalNac4 | Covalent bond | Oligo 4 | 4,151 |
| (SEQ. ID NO: 10) | | | | |
| Conjugate 4 | GalNac6 | Covalent bond | Oligo 4 | 5,473 |
| (SEQ. ID NO: 11) | | | | |
| Conjugate 5 | GalNac4-Oligo 4 | Covalent bond | GalNac4 | 482 |
| (SEQ. ID NO: 12) | | | | |
| Conjugate 6 | Cholesterol | poHEGpo | Oligo 4 | 8,808 |
| (SEQ. ID NO: 13) | | | | |
| Conjugate 7 | GalNac2 | Hexyl | Oligo 4 | 14,767 |
| (SEQ. ID NO: 14) | | | | |
| Conjugate 8 | GalNac4 | poApo | Oligo 4 | 8,778 |
| (SEQ. ID NO: 15) | | | | |
| Conjugate 9 | GalNac4 | poApoApo | Oligo 4 | 10,670 |
| (SEQ. ID NO: 16) | | | | |
| Conjugate 10 | GalNac4 | poApoApoApo | Oligo 2 | 12,130 |
| (SEQ. ID NO: 17) | | | | |
| Conjugate 11 | GalNac4 | poApoApoApo | Oligo 4 | 13,150 |
| (SEQ. ID NO: 18) | | | | |
| Conjugate 12 | GalNac4 | poHEGpo | Oligo 4 | 7,747 |
| (SEQ. ID NO: 19) | | | | |
| Conjugate 13 | GalNac4 | poHEGpoHEGpo | Oligo 4 | 6,682 |
| (SEQ. ID NO: 20) | | | | |
| Conjugate 14 | GalNac4 | pomApoln(5m)C- | Oligo 4 | 754 |
| (SEQ. ID NO: 21) | | pomApoln(5m)Cpo | | |
| Conjugate 15 | GalNac4 | pomApomApomApo | Oligo 4 | 1,025 |
| (SEQ. ID NO: 22) | | | | |
| Conjugate 16 | GalNac4 | porAporAporApo | Oligo 4 | 13,651 |
| (SEQ. ID NO: 23) | | | | |
| Conjugate 17 | GalNac6 | poApoApoApo | Oligo 4 | 8,133 |
| (SEQ. ID NO: 24) | | | | |
| Conjugate 18 | Palmitoyl | poApoApoApo | Oligo 4 | 10,793 |
| (SEQ. ID NO: 25) | | | | |
| Conjugate 19 | Palmitoyl | poHEGpo | Oligo 4 | 16,575 |
| (SEQ. ID NO: 26) | | | | |

### Examples B20-B25

Oligonucleotide conjugates were administered subcutaneously to the interscapular region of CD-1 mice (Charles River, n=4) at a dosage of 15 mg/kg using standard methods. The resulting concentrations of the modified oligonucleotides in the livers of the test subjects at the 24 hour timepoint after administration were determined using standard LC-MS/MS methods, as summarized in Table 6.

TABLE 6

| | | | | Oligo Conc.In |
|---|---|---|---|---|
| Conjugate No. | 5' end | Linker | 3' end | Liver (ng/g) |
| Conjugate 20 | Oligo 1 | Covalent bond | Ga1Nac2 | 20,138 |
| (SEQ. ID NO:27) | | | | |
| Conjugate 21 | GalNac2 | Covalent bond | Oligo 1 | 56,339 |
| (SEQ. ID NO: 28) | | | | |
| Conjugate 22 | GalNac4 | Covalent bond | Oligo 1 | 5,838 |
| (SEQ. ID NO: 29) | | | | |
| Conjugate 23 | Oligo 1 | poCpoApo | GalNac2 | 42,804 |
| (SEQ. ID NO: 30) | | | | |
| Conjugate 24 | GalNac2 | poCpoApo | Oligo 1 | 49,411 |
| (SEQ. ID NO: 31) | | | | |
| Conjugate 27 | GalNac4 | poApoApoApo | Oligo 3 | 18,024 |
| (SEQ. ID NO: 34) | | | | |

### Examples C1-C4

Oligonucleotides (both conjugated and unconjugated) were administered subcutaneously to the neck or back of non-human primates (n=2) at a dosage of 5 mg/kg (Oligo 3, Oligo 4 and Conjugate 11) or 6.1 mg/kg (Conjugate 27) using standard methods. The resulting concentrations of the modified oligonucleotides in the livers of the test subjects at the 48 hour timepoint after administration were determined using standard LC-MS/MS methods, as summarized in Table 7.

TABLE 7

| Oligo or | | | | Oligo Conc. |
|---|---|---|---|---|
| Conjugate No. | 5' end | Linker | 3' end | In Liver(ng/g) |
| Oligo 3 | None | None | Oligo 3 | 14,200 |
| (SEQ ID NO: 3) | | | | |
| Conjugate 27 | Ga1Nac4 | poApoApoApo | Oligo 3 | 33,350 |
| (SEQ ID NO: 34) | | | | |
| Oligo 4 | None | None | Oligo 4 | 44,400 |
| (SEQ ID NO: 4) | | | | |
| Conjugate 11 | Ga1Nac4 | poApoApoApo | Oligo 4 | 75,350 |
| (SEQ ID NO: 18) | | | | |

Unexpectedly (see WO 2016/030863), it has now been found that improved targeting of a modified oligonucleotide to the liver can be obtained by linking the modified oligonucleotide to a liver targeting agent using a linker as described herein. For example, as illustrated by the data in Table 8, the liver targeting of Oligo 4 (SEQ ID NO: 4) was enhanced by more than 50% by conjugating to a GalNac4 targeting agent using a poApo spacer (Conjugate 8, SEQ ID NO: 15), a poApoApo spacer (Conjugate 9, SEQ ID NO: 16) or a poApoApoApo spacer (Conjugate 11, SEQ ID NO: 18), as compared to a direct linkage via covalent bond (Conjugate 3, SEQ ID NO: 10).

TABLE 8

| | | | | Oligo Conc. In |
|---|---|---|---|---|
| Conjugate No. | 5' end | Linker | 3' end | Liver (ng/g) |
| Conjugate 3 | GalNac4 | Covalent bond | Oligo 4 | 4,151 |
| (SEQ. ID NO: 10) | | | | |
| Conjugate 8 | GalNac4 | poApo | Oligo 4 | 8,778 |
| (SEQ. ID NO: 15) | | | | |
| Conjugate 9 | GalNac4 | poApoApo | Oligo 4 | 10,670 |
| (SEQ. ID NO: 16) | | | | |
| Conjugate 11 | GalNac4 | poApoApoApo | Oligo 4 | 13,150 |
| (SEQ. ID NO: 18) | | | | |

### Examples D1-D6

Oligonucleotides (both unconjugated and conjugated to GalNac4) were administered subcutaneously to the interscapular region of CD-1 mice (Charles River, n=4) at a dosage of between 5 and 6.1 mg/kg, using standard methods. The resulting concentrations of the modified oligonucleotides in the livers and kidneys of the test subjects at the 24 hour timepoint after administration were determined using standard LC-MS/MS methods, as summarized in Table 9.

TABLE 9

| | | Mean ± SD | Mean ± SD | |
|---|---|---|---|---|
| Oligo or Conjugate | Dose | Liver conc. | Kidney conc. | Liver: |
| No. | (mg/kg) | (ng/g) | (ng/g) | Kidney Ratio |
| Conjugate 47 (Oligo | | | | |
| 6-Ga1Nac4) | 5.8 | 744 ± 149 | Not detected | — |
| (SEQ. ID NO: 54) | | | | |
| Conjugate 48 (Oligo | | | | |
| 6-AAA-Ga1Nac4) | 6.1 | 2,653 ± 998 | Not detected | — |
| (SEQ. ID NO: 55) | | | | |
| Oligo 7 | | | | |
| (SEQ. ID NO:7) | 5 | 2,905 ± 365 | 5,105 ± 972 | 0.6 |
| Conjugate 49 (Oligo | | | | |
| 7-Ga1Nac4) | 5.8 | 855 ± 821 | Not detected | — |
| (SEQ. ID NO: 56) | | | | |
| Conjugate 50 | | | | |
| (Oligo-AAA-GalNac4) | 6.1 | 4,655 ± 830 | Not detected | — |
| (SEQ. ID NO:57) | | | | |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. An oligonucleotide conjugate or complex thereof, comprising:
a modified oligonucleotide of 30 to 50 nucleotides in length and having sequence independent antiviral activity against hepatitis B;
a liver targeting agent; and
a linker connecting the liver targeting agent to a 3′ end and/or a 5′ end of the modified oligonucleotide or complex thereof;
wherein:
each liver targeting agent is independently GalNac1, GalNac2, GalNac3, GalNac4, GalNac5, GalNac6, a cholesterol, a tocopherol, a C₁₂₋₂₀ alkyl ester or a C₁₂₋₂₀ alkenyl ester;
each linker is independently C₄₋₈ alkyl, -po-(Nuc-po)ₓ-, -po-O-(CH₂CH₂O)ₘ-po- or -po[-O-(CH₂CH₂O)ₘ-po-O-(CH₂CH₂O)ₙ]-po-;
each Nuc in -po-(Nuc-po)ₓ- is independently an A nucleotide or a C nucleotide;
each A nucleotide in -po-(Nuc-po)ₓ- is independently adenosine, 2′-O-methyladenosine, or deoxyadenosine;
each C nucleotide in -po-(Nuc-po)ₓ- is independently cytidine, 2′-O-methylcytidine, a locked/bridged cytidine, a locked/bridged methylcytidine, ribose cytidine, or deoxycytidine;
po is phosphodiester;
each x is independently an integer in the range of 1 to 5; and
m and n are each independently an integer in the range of 2 to 10.

2. The oligonucleotide conjugate or complex thereof of claim 1, wherein the liver targeting agent and the linker are selected to provide improved targeting of the modified oligonucleotide to the liver of a mouse, non-human primate, human, or other mammalian subject to which the oligonucleotide conjugate or complex thereof is administered, and wherein the improved targeting is determined by comparison to a corresponding modified oligonucleotide conjugate that lacks the spacer, at a 6-hour, 24-hour, or 48-hour timepoint following subcutaneous administration to a mouse, non-human primate, human, or other mammalian subject at a dosage of about 0.05, 0.1, 0.15, 0.25, 0.5, 0.75, 1, 2, 3, 4, 5, 6, 7, 8, 9, 10, 11, 12, 13, 14 or 15 mg/kg, or any range that is defined by two of the aforementioned values as endpoints.

3. The oligonucleotide conjugate or complex thereof of claim 2, wherein the improved targeting is evidenced by a liver concentration of the modified oligonucleotide that is at least about 8,000 ng/g, at least about 9,000 ng/g, at least about 10,000 ng/g, at least about 11,000 ng/g, at least about 12,000 ng/g, at least about 13,000 ng/g, at least about 18,000 ng/g, at least about 40,000 ng/g, at least about 50,000 ng/g, or at least about 60,000 ng/g.

4. The oligonucleotide conjugate or complex thereof of claim 1, wherein the liver targeting agent is GalNac1.

5. The oligonucleotide conjugate or complex thereof of claim 1, wherein the liver targeting agent is GalNac2.

6. The oligonucleotide conjugate or complex thereof of claim 1, wherein the liver targeting agent is GalNac3.

7. The oligonucleotide conjugate or complex thereof of claim 1, wherein the liver targeting agent is GalNac4.

8. The oligonucleotide conjugate or complex thereof of claim 1, wherein the liver targeting agent is GalNac5.

9. The oligonucleotide conjugate or complex thereof of claim 1, wherein the liver targeting agent is GalNac6.

10. The oligonucleotide conjugate or complex thereof of claim 1, wherein the liver targeting agent is a cholesterol.

11. The oligonucleotide conjugate or complex thereof of claim 1, wherein the liver targeting agent is a tocopherol.

12. The oligonucleotide conjugate or complex thereof of claim 1, wherein the liver targeting agent is a C₁₂₋₂₀ alkyl or alkenyl ester.

13. The oligonucleotide conjugate or complex thereof of claim 12, wherein the C₁₂₋₂₀ alkyl or alkenyl ester is a palmitate or palmitoyl.

14. The oligonucleotide conjugate or complex thereof of claim 1, wherein the linker is -po-(Nuc-po)ₓ-.

15. The oligonucleotide conjugate or complex thereof of claim 14, wherein at least one A nucleotide in -po-(Nuc-po)ₓ- is adenosine.

16. The oligonucleotide conjugate or complex thereof of claim 14, wherein at least one C nucleotide in -po-(Nuc-po)ₓ- is cytidine.

17. The oligonucleotide conjugate or complex thereof of claim 14, wherein at least one nucleotide in -po-(Nuc-po)ₓ- is a deoxy-nucleotide.

18. The oligonucleotide conjugate or complex thereof of claim 14, wherein at least one nucleotide in -po-(Nuc-po)ₓ- is a 2′-O-methylated nucleotide.

19. The oligonucleotide conjugate or complex thereof of claim 14, wherein at least one nucleotide in -po-(Nuc-po)ₓ- is a ribo-nucleotide.

20. The oligonucleotide conjugate or complex thereof of claim 14, wherein at least one C nucleotide in -po-(Nuc-po)ₓ- is 5-methylcytidine.

21. The oligonucleotide conjugate or complex thereof of claim 14, wherein at least one C nucleotide in -po-(Nuc-po)ₓ- is 2′-O-methyl-5-methylcytidine.

22. The oligonucleotide conjugate or complex thereof of claim 14, wherein at least one C nucleotide in the linker is a locked/bridged cytidine or a locked/bridged methylcytidine.

23. The oligonucleotide conjugate or complex thereof of claim 14, wherein x is 1.

24. The oligonucleotide conjugate or complex thereof of claim 14, wherein x is 2.

25. The oligonucleotide conjugate or complex thereof of claim 14, wherein x is 3.

26. The oligonucleotide conjugate or complex thereof of claim 14, wherein x is 4 or 5.

27. The oligonucleotide conjugate or complex thereof of claim 14, wherein the linker is either -po-(C-po)-(A-po)ₓ₋₁- or -po-(A-po)-(C-po)ₓ₋₁-.

28. The oligonucleotide conjugate or complex thereof of claim 27, wherein x is 1.

29. The oligonucleotide conjugate or complex thereof of claim 27, wherein x is 2.

30. The oligonucleotide conjugate or complex thereof of claim 27, wherein x is 3.

31. The oligonucleotide conjugate or complex thereof of claim 27, wherein x is 4 or 5.

32. The oligonucleotide conjugate or complex thereof of claim 1, wherein the linker is -po-O-(CH₂CH₂O)ₘ-po-.

33. The oligonucleotide conjugate or complex thereof of claim 1, wherein the linker is -po[-O-(CH₂CH₂O)ₘ-po-O-(CH₂CH₂O)ₙ]-po-.

34. The oligonucleotide conjugate or complex thereof of claim 32, wherein m is 2, 3, or 4.

35. The oligonucleotide conjugate or complex thereof of claim 32, wherein m is 5, 6 or 7.

36. The oligonucleotide conjugate or complex thereof of claim 30, wherein m is 8, 9 or 10.

37. The oligonucleotide conjugate or complex thereof of claim 33, wherein n is 2, 3 or 4.

38. The oligonucleotide conjugate or complex thereof of claim 32, wherein n is 5, 6 or 7.

39. The oligonucleotide conjugate or complex thereof of claim 30, wherein n is 8, 9 or 10.

40. The oligonucleotide conjugate or complex thereof of claim 1, wherein the linker is C₄₋₈ alkyl.

41. The oligonucleotide conjugate or complex thereof of claim 40, wherein the linker is hexyl.

42. The oligonucleotide conjugate or complex thereof of claim 1, wherein the linker is poApoApoApo.

43. An oligonucleotide conjugate or complex thereof, wherein the oligonucleotide conjugate comprises Oligo 4 (SEQ ID NO: 4) attached to GalNac4 via poApoApoApo linker.

44. The oligonucleotide conjugate or complex thereof of claim 43, wherein the oligonucleotide conjugate is Conjugate 11 (SEQ ID NO: 18).

45. An oligonucleotide conjugate or complex thereof, wherein the oligonucleotide conjugate comprises Oligo 3 (SEQ ID NO: 3) attached to GalNac4 via poApoApoApo linker.

46. The oligonucleotide conjugate or complex thereof of claim 45, wherein the oligonucleotide conjugate is Conjugate 27 (SEQ ID NO: 18).

47. An oligonucleotide conjugate or complex thereof, wherein the oligonucleotide conjugate comprises Oligo 2 (SEQ ID NO: 2) attached to a liver targeting agent via a linker.

48. The oligonucleotide conjugate or complex thereof of claim 47, wherein the liver targeting agent is GalNac4.

49. The oligonucleotide conjugate or complex thereof of claim 47, wherein the linker is poApoApoApo.

50. The oligonucleotide conjugate or complex thereof of claim 47, wherein the oligonucleotide conjugate is Conjugate 10 (SEQ ID NO: 17).

51. An oligonucleotide conjugate or complex thereof, wherein the oligonucleotide conjugate comprises Oligo 6 (SEQ ID NO: 6) attached to a liver targeting agent via a linker.

52. The oligonucleotide conjugate or complex thereof of claim 51, wherein the liver targeting agent is GalNac4.

53. The oligonucleotide conjugate or complex thereof of claim 51, wherein the linker is poApoApoApo.

54. The oligonucleotide conjugate or complex thereof of claim 51, wherein the oligonucleotide conjugate is Conjugate 47 (SEQ ID NO: 54).

55. The oligonucleotide conjugate or complex thereof of claim 51, wherein the oligonucleotide conjugate is Conjugate 48 (SEQ ID NO: 55).

56. An oligonucleotide conjugate or complex thereof, wherein the oligonucleotide conjugate comprises Oligo 7 (SEQ ID NO: 7) attached to a liver targeting agent via a linker.

57. The oligonucleotide conjugate or complex thereof of claim 56, wherein the liver targeting agent is GalNac4.

58. The oligonucleotide conjugate or complex thereof of claim 56, wherein the linker is poApoApoApo.

59. The oligonucleotide conjugate or complex thereof of claim 56, wherein the oligonucleotide conjugate is Conjugate 49 (SEQ ID NO: 56).

60. The oligonucleotide conjugate or complex thereof of claim 56, wherein the oligonucleotide conjugate is Conjugate 50 (SEQ ID NO: 57).

61. The oligonucleotide conjugate or complex thereof of claim 1, wherein the oligonucleotide conjugate is selected from any one of Conjugates 1 to 50 (SEQ ID NOS: 7 to 57) as provided in Table 3.

62. The complex of the oligonucleotide conjugate of claim 1, wherein the complex is a chelate complex.

63. The complex of claim 62, wherein the complex is a monovalent counterion complex of the oligonucleotide conjugate.

64. The complex of claim 63, wherein the complex is a lithium, sodium or potassium complex of the oligonucleotide conjugate.

65. A pharmaceutical composition for treating a liver disease or disorder in a subject, comprising:
a pharmaceutically acceptable carrier, diluent, excipient or combination thereof; and
a therapeutically effective amount of the oligonucleotide conjugate or complex thereof of claim 1.

66. The pharmaceutical composition of claim 65, wherein the pharmaceutical composition is in a form suitable for administration to a subject via parenteral delivery.

67. The pharmaceutical composition of claim 66, wherein the parenteral delivery comprises intravenous delivery.

68. The pharmaceutical composition of claim 66, wherein the parenteral delivery comprises subcutaneous delivery.

69. The pharmaceutical composition of claims 65, wherein the liver disease or disorder is hepatitis B.

70. The pharmaceutical composition of claim 65, wherein the liver disease or disorder is hepatitis D.

71. The pharmaceutical composition of claim 65, wherein the liver disease or disorder is a liver cirrhosis that is developed because of a Hepatitis B infection.

72. A method of treating a liver disease or disorder, comprising:
administering an effective amount of the oligonucleotide conjugate or complex thereof of claim 1, to a subject in need thereof.

73. The method of claim 72, wherein at least a portion of the effective amount of the oligonucleotide conjugate or complex thereof is administered to the subject parenterally.

74. The method of claim 73, wherein at least a portion of the effective amount of the oligonucleotide conjugate or complex thereof is administered to the subject intravenously.

75. The method of claim 73, wherein at least a portion of the effective amount of the oligonucleotide conjugate or complex thereof is administered to the subject subcutaneously.

76. The method of claim 72, wherein the subject is a human.

77. The method of claim 72, further comprising administering an effective amount of at least one second therapeutic molecule to the subject in combination with the oligonucleotide conjugate or complex thereof.

78. The method of claim 77, wherein the at least one second therapeutic molecule comprises a second oligonucleotide conjugate or complex thereof, an oligonucleotide inhibitor, a nucleoside, a nucleotide, a nucleotide prodrug, an interferon, a capsid assembly modulator, or a combination thereof.

79. The method of claim 72, wherein the liver disease or disorder is Hepatitis B.

80. The method of claim 72, wherein the liver disease or disorder is Hepatitis D.

81. The method of claim 72, wherein the liver disease or disorder is a liver cirrhosis that is developed because of a Hepatitis B infection.


SEQUENCE LISTING (57 sequences)

SEQ ID NO: 1 - DNA, 40 bp, Artificial Sequence
  Modified Oligonucleotide
  misc_feature (1)...(40): phosphorothioate internucleoside linkages
  misc_feature (1)...(40): LNA nucleoside
  misc_feature (2)...(2): LNA C is 5-methyl C
  misc_feature (4)...(4): LNA C is 5-methyl C
  misc_feature (6)...(6): LNA C is 5-methyl C
  misc_feature (8)...(8): LNA C is 5-methyl C
  misc_feature (10)...(10): LNA C is 5-methyl C
  misc_feature (12)...(12): LNA C is 5-methyl C
  misc_feature (14)...(14): LNA C is 5-methyl C
  misc_feature (16)...(16): LNA C is 5-methyl C
  misc_feature (18)...(18): LNA C is 5-methyl C
  misc_feature (20)...(20): LNA C is 5-methyl C
  misc_feature (22)...(22): LNA C is 5-methyl C
  misc_feature (24)...(24): LNA C is 5-methyl C
  misc_feature (26)...(26): LNA C is 5-methyl C
  misc_feature (28)...(28): LNA C is 5-methyl C
  misc_feature (30)...(30): LNA C is 5-methyl C
  misc_feature (32)...(32): LNA C is 5-methyl C
  misc_feature (34)...(34): LNA C is 5-methyl C
  misc_feature (36)...(36): LNA C is 5-methyl C
  misc_feature (38)...(38): LNA C is 5-methyl C
  misc_feature (40)...(40): LNA C is 5-methyl C
acacacacacacacacacacacacacacacacacacacac

SEQ ID NO: 2 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): mA is 2′-O-Methyladenosine
  misc_feature (2)...(2): m(5m)C is 2′-0, 5- dimethylcytidine
  misc_feature (3)...(3): mA is 2′-O-Methyladenosine
  misc_feature (4)...(4): m(5m)C is 2′-0, 5- dimethylcytidine
  misc_feature (5)...(5): mA is 2′-O-Methyladenosine
  misc_feature (6)...(6): m(5m)C is 2′-0, 5- dimethylcytidine
  misc_feature (7)...(7): mA is 2′-O-Methyladenosine
  misc_feature (8)...(8): m(5m)C is 2′-0, 5- dimethylcytidine
  misc_feature (9)...(9): mA is 2′-O-Methyladenosine
  misc_feature (10)...(10): m(5m)C is 2′-0, 5- dimethylcytidine
  misc_feature (11)...(11): mA is 2′-O-Methyladenosine
  misc_feature (12)...(12): m(5m)C is 2′-0, 5- dimethylcytidine
  misc_feature (13)...(13): mA is 2′-O-Methyladenosine
  misc_feature (14)...(14): m(5m)C is 2′-0, 5- dimethylcytidine
  misc_feature (15)...(15): mA is 2′-O-Methyladenosine
  misc_feature (16)...(16): m(5m)C is 2′-0, 5- dimethylcytidine
  misc_feature (17)...(17): mA is 2′-O-Methyladenosine
  misc_feature (18)...(18): m(5m)C is 2′-0, 5- dimethylcytidine
  misc_feature (19)...(19): mA is 2′-O-Methyladenosine
  misc_feature (20)...(20): m(5m)C is 2′-0, 5- dimethylcytidine
  misc_feature (21)...(21): mA is 2′-O-Methyladenosine
  misc_feature (22)...(22): m(5m)C is 2′-0, 5- dimethylcytidine
  misc_feature (23)...(23): mA is 2′-O-Methyladenosine
  misc_feature (24)...(24): m(5m)C is 2′-0, 5- dimethylcytidine
  misc_feature (25)...(25): mA is 2′-O-Methyladenosine
  misc_feature (26)...(26): m(5m)C is 2′-0, 5- dimethylcytidine
  misc_feature (27)...(27): mA is 2′-O-Methyladenosine
  misc_feature (28)...(28): m(5m)C is 2′-0, 5- dimethylcytidine
  misc_feature (29)...(29): mA is 2′-O-Methyladenosine
  misc_feature (30)...(30): m(5m)C is 2′-0, 5- dimethylcytidine
  misc_feature (31)...(31): mA is 2′-O-Methyladenosine
  misc_feature (32)...(32): m(5m)C is 2′-0, 5- dimethylcytidine
  misc_feature (33)...(33): mA is 2′-O-Methyladenosine
  misc_feature (34)...(34): m(5m)C is 2′-0, 5- dimethylcytidine
  misc_feature (35)...(35): mA is 2′-O-Methyladenosine
  misc_feature (36)...(36): m(5m)C is 2′-0, 5- dimethylcytidine
  misc_feature (37)...(37): mA is 2′-O-Methyladenosine
  misc_feature (38)...(38): m(5m)C is 2′-0, 5- dimethylcytidine
  misc_feature (39)...(39): mA is 2′-O-Methyladenosine
  misc_feature (40)...(40): m(5m)C is 2′-0, 5- dimethylcytidine

SEQ ID NO: 3 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 4 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 5 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): B is 1-benzyl, 2-O-methylriboside
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): B is 1-benzyl, 2-O-methylriboside
  misc_feature (11)...(11): mA is 2′-O-Methyladenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): B is 1-benzyl, 2-O-methylriboside
  misc_feature (17)...(17): mA is 2′-O-Methyladenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): B is 1-benzyl, 2-O-methylriboside
  misc_feature (23)...(23): mA is 2′-O-Methyladenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): B is 1-benzyl, 2-O-methylriboside
  misc_feature (29)...(29): mA is 2′-O-Methyladenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): B is 1-benzyl, 2-O-methylriboside
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 6 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): mA is 2′-O-Methyladenosine
  misc_feature (2)...(2): m(5m)C is 2′-0, 5- dimethylcytidine
  misc_feature (3)...(3): mA is 2′-O-Methyladenosine
  misc_feature (4)...(4): m(5m)C is 2′-0, 5- dimethylcytidine
  misc_feature (5)...(5): mA is 2′-O-Methyladenosine
  misc_feature (6)...(6): m(5m)C is 2′-0, 5- dimethylcytidine
  misc_feature (7)...(7): mA is 2′-O-Methyladenosine
  misc_feature (8)...(8): m(5m)C is 2′-0, 5- dimethylcytidine
  misc_feature (9)...(9): mA is 2′-O-Methyladenosine
  misc_feature (10)...(10): m(5m)C is 2′-0, 5- dimethylcytidine
  misc_feature (11)...(11): rA is ribo-adenosine
  misc_feature (12)...(12): m(5m)C is 2′-0, 5- dimethylcytidine
  misc_feature (13)...(13): mA is 2′-O-Methyladenosine
  misc_feature (14)...(14): m(5m)C is 2′-0, 5- dimethylcytidine
  misc_feature (15)...(15): mA is 2′-O-Methyladenosine
  misc_feature (16)...(16): m(5m)C is 2′-0, 5- dimethylcytidine
  misc_feature (17)...(17): mA is 2′-O-Methyladenosine
  misc_feature (18)...(18): m(5m)C is 2′-0, 5- dimethylcytidine
  misc_feature (19)...(19): mA is 2′-O-Methyladenosine
  misc_feature (20)...(20): m(5m)C is 2′-0, 5- dimethylcytidine
  misc_feature (21)...(21): rA is ribo-adenosine
  misc_feature (22)...(22): m(5m)C is 2′-0, 5- dimethylcytidine
  misc_feature (23)...(23): mA is 2′-O-Methyladenosine
  misc_feature (24)...(24): m(5m)C is 2′-0, 5- dimethylcytidine
  misc_feature (25)...(25): mA is 2′-O-Methyladenosine
  misc_feature (26)...(26): m(5m)C is 2′-0, 5- dimethylcytidine
  misc_feature (27)...(27): mA is 2′-O-Methyladenosine
  misc_feature (28)...(28): m(5m)C is 2′-0, 5- dimethylcytidine
  misc_feature (29)...(29): mA is 2′-O-Methyladenosine
  misc_feature (30)...(30): m(5m)C is 2′-0, 5- dimethylcytidine
  misc_feature (31)...(31): rA is ribo-adenosine
  misc_feature (32)...(32): m(5m)C is 2′-0, 5- dimethylcytidine
  misc_feature (33)...(33): mA is 2′-O-Methyladenosine
  misc_feature (34)...(34): m(5m)C is 2′-0, 5- dimethylcytidine
  misc_feature (35)...(35): mA is 2′-O-Methyladenosine
  misc_feature (36)...(36): m(5m)C is 2′-0, 5- dimethylcytidine
  misc_feature (37)...(37): mA is 2′-O-Methyladenosine
  misc_feature (38)...(38): m(5m)C is 2′-0, 5- dimethylcytidine
  misc_feature (39)...(39): mA is 2′-O-Methyladenosine
  misc_feature (40)...(40): m(5m)C is 2′-0, 5- dimethylcytidine

SEQ ID NO: 7 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): lnA is LNA adenosine
  misc_feature (2)...(2): m(5m)C is 2′-0, 5- dimethylcytidine
  misc_feature (3)...(3): mA is 2′-O-Methyladenosine
  misc_feature (4)...(4): m(5m)C is 2′-0, 5- dimethylcytidine
  misc_feature (5)...(5): mA is 2′-O-Methyladenosine
  misc_feature (6)...(6): m(5m)C is 2′-0, 5- dimethylcytidine
  misc_feature (7)...(7): mA is 2′-O-Methyladenosine
  misc_feature (8)...(8): m(5m)C is 2′-0, 5- dimethylcytidine
  misc_feature (9)...(9): mA is 2′-O-Methyladenosine
  misc_feature (10)...(10): m(5m)C is 2′-0, 5- dimethylcytidine
  misc_feature (11)...(11): rA is ribo-adenosine
  misc_feature (12)...(12): m(5m)C is 2′-0, 5- dimethylcytidine
  misc_feature (13)...(13): mA is 2′-O-Methyladenosine
  misc_feature (14)...(14): m(5m)C is 2′-0, 5- dimethylcytidine
  misc_feature (15)...(15): mA is 2′-O-Methyladenosine
  misc_feature (16)...(16): m(5m)C is 2′-0, 5- dimethylcytidine
  misc_feature (17)...(17): mA is 2′-O-Methyladenosine
  misc_feature (18)...(18): m(5m)C is 2′-0, 5- dimethylcytidine
  misc_feature (19)...(19): mA is 2′-O-Methyladenosine
  misc_feature (20)...(20): m(5m)C is 2′-0, 5- dimethylcytidine
  misc_feature (21)...(21): rA is ribo-adenosine
  misc_feature (22)...(22): m(5m)C is 2′-0, 5- dimethylcytidine
  misc_feature (23)...(23): mA is 2′-O-Methyladenosine
  misc_feature (24)...(24): m(5m)C is 2′-0, 5- dimethylcytidine
  misc_feature (25)...(25): mA is 2′-O-Methyladenosine
  misc_feature (26)...(26): m(5m)C is 2′-0, 5- dimethylcytidine
  misc_feature (27)...(27): mA is 2′-O-Methyladenosine
  misc_feature (28)...(28): m(5m)C is 2′-0, 5- dimethylcytidine
  misc_feature (29)...(29): mA is 2′-O-Methyladenosine
  misc_feature (30)...(30): m(5m)C is 2′-0, 5- dimethylcytidine
  misc_feature (31)...(31): rA is ribo-adenosine
  misc_feature (32)...(32): m(5m)C is 2′-0, 5- dimethylcytidine
  misc_feature (33)...(33): mA is 2′-O-Methyladenosine
  misc_feature (34)...(34): m(5m)C is 2′-0, 5- dimethylcytidine
  misc_feature (35)...(35): mA is 2′-O-Methyladenosine
  misc_feature (36)...(36): m(5m)C is 2′-0, 5- dimethylcytidine
  misc_feature (37)...(37): mA is 2′-O-Methyladenosine
  misc_feature (38)...(38): m(5m)C is 2′-0, 5- dimethylcytidine
  misc_feature (39)...(39): mA is 2′-O-Methyladenosine
  misc_feature (40)...(40): LNA nucleoside, LNA C is 5-methyl C

SEQ ID NO: 8 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C
  misc_feature (40)...(40): 3 prime GalNAc4 conjugate

SEQ ID NO: 9 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime cholesterol conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 10 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4 conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 11 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6 conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 12 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4 conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C
  misc_feature (40)...(40): 3 prime GalNac4 conjugate

SEQ ID NO: 13 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime cholesterol-po-HEG-po conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 14 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac2-hexyl conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 15 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poApo- conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 16 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poApoApo- conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 17 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poApoApoApo conjugate
  misc_feature (1)...(1): mA is 2′-O-Methyladenosine
  misc_feature (2)...(2): m(5m)C is 2′-0, 5- dimethylcytidine
  misc_feature (3)...(3): mA is 2′-O-Methyladenosine
  misc_feature (4)...(4): m(5m)C is 2′-0, 5- dimethylcytidine
  misc_feature (5)...(5): mA is 2′-O-Methyladenosine
  misc_feature (6)...(6): m(5m)C is 2′-0, 5- dimethylcytidine
  misc_feature (7)...(7): mA is 2′-O-Methyladenosine
  misc_feature (8)...(8): m(5m)C is 2′-0, 5- dimethylcytidine
  misc_feature (9)...(9): mA is 2′-O-Methyladenosine
  misc_feature (10)...(10): m(5m)C is 2′-0, 5- dimethylcytidine
  misc_feature (11)...(11): mA is 2′-O-Methyladenosine
  misc_feature (12)...(12): m(5m)C is 2′-0, 5- dimethylcytidine
  misc_feature (13)...(13): mA is 2′-O-Methyladenosine
  misc_feature (14)...(14): m(5m)C is 2′-0, 5- dimethylcytidine
  misc_feature (15)...(15): mA is 2′-O-Methyladenosine
  misc_feature (16)...(16): m(5m)C is 2′-0, 5- dimethylcytidine
  misc_feature (17)...(17): mA is 2′-O-Methyladenosine
  misc_feature (18)...(18): m(5m)C is 2′-0, 5- dimethylcytidine
  misc_feature (19)...(19): mA is 2′-O-Methyladenosine
  misc_feature (20)...(20): m(5m)C is 2′-0, 5- dimethylcytidine
  misc_feature (21)...(21): mA is 2′-O-Methyladenosine
  misc_feature (22)...(22): m(5m)C is 2′-0, 5- dimethylcytidine
  misc_feature (23)...(23): mA is 2′-O-Methyladenosine
  misc_feature (24)...(24): m(5m)C is 2′-0, 5- dimethylcytidine
  misc_feature (25)...(25): mA is 2′-O-Methyladenosine
  misc_feature (26)...(26): m(5m)C is 2′-0, 5- dimethylcytidine
  misc_feature (27)...(27): mA is 2′-O-Methyladenosine
  misc_feature (28)...(28): m(5m)C is 2′-0, 5- dimethylcytidine
  misc_feature (29)...(29): mA is 2′-O-Methyladenosine
  misc_feature (30)...(30): m(5m)C is 2′-0, 5- dimethylcytidine
  misc_feature (31)...(31): mA is 2′-O-Methyladenosine
  misc_feature (32)...(32): m(5m)C is 2′-0, 5- dimethylcytidine
  misc_feature (33)...(33): mA is 2′-O-Methyladenosine
  misc_feature (34)...(34): m(5m)C is 2′-0, 5- dimethylcytidine
  misc_feature (35)...(35): mA is 2′-O-Methyladenosine
  misc_feature (36)...(36): m(5m)C is 2′-0, 5- dimethylcytidine
  misc_feature (37)...(37): mA is 2′-O-Methyladenosine
  misc_feature (38)...(38): m(5m)C is 2′-0, 5- dimethylcytidine
  misc_feature (39)...(39): mA is 2′-O-Methyladenosine
  misc_feature (40)...(40): m(5m)C is 2′-0, 5- dimethylcytidine

SEQ ID NO: 18 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poApoApoApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 19 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poHEGpo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 20 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poHEGpoHEGpo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 21 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-pomApoln(5m)CpomApoln(5m)Cpo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 22 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-pomApomApomApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 23 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-porAporAporApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 24 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-poApoApoApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 25 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime palmitoyl-poApoApoApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 26 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime palmitoyl-poHEGpo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): mA is 2′-O-Methyladenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 27 - DNA, 40 bp, Artificial Sequence; identical to SEQ ID NO: 1
  Modified Oligonucleotide
  misc_feature (1)...(40): phosphorothioate internucleoside linkages
  misc_feature (1)...(40): LNA nucleoside
  misc_feature (2)...(2): LNA C is 5-methyl C
  misc_feature (4)...(4): LNA C is 5-methyl C
  misc_feature (6)...(6): LNA C is 5-methyl C
  misc_feature (8)...(8): LNA C is 5-methyl C
  misc_feature (10)...(10): LNA C is 5-methyl C
  misc_feature (12)...(12): LNA C is 5-methyl C
  misc_feature (14)...(14): LNA C is 5-methyl C
  misc_feature (16)...(16): LNA C is 5-methyl C
  misc_feature (18)...(18): LNA C is 5-methyl C
  misc_feature (20)...(20): LNA C is 5-methyl C
  misc_feature (22)...(22): LNA C is 5-methyl C
  misc_feature (24)...(24): LNA C is 5-methyl C
  misc_feature (26)...(26): LNA C is 5-methyl C
  misc_feature (28)...(28): LNA C is 5-methyl C
  misc_feature (30)...(30): LNA C is 5-methyl C
  misc_feature (32)...(32): LNA C is 5-methyl C
  misc_feature (34)...(34): LNA C is 5-methyl C
  misc_feature (36)...(36): LNA C is 5-methyl C
  misc_feature (38)...(38): LNA C is 5-methyl C
  misc_feature (40)...(40): LNA C is 5-methyl C
  misc_feature (40)...(40): 3 prime GalNac2 conjugate

SEQ ID NO: 28 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac2 conjugate
  misc_feature (1)...(40): LNA nucleoside
  misc_feature (2)...(2): LNA C is 5-methyl C
  misc_feature (4)...(4): LNA C is 5-methyl C
  misc_feature (6)...(6): LNA C is 5-methyl C
  misc_feature (8)...(8): LNA C is 5-methyl C
  misc_feature (10)...(10): LNA C is 5-methyl C
  misc_feature (12)...(12): LNA C is 5-methyl C
  misc_feature (14)...(14): LNA C is 5-methyl C
  misc_feature (16)...(16): LNA C is 5-methyl C
  misc_feature (18)...(18): LNA C is 5-methyl C
  misc_feature (20)...(20): LNA C is 5-methyl C
  misc_feature (22)...(22): LNA C is 5-methyl C
  misc_feature (24)...(24): LNA C is 5-methyl C
  misc_feature (26)...(26): LNA C is 5-methyl C
  misc_feature (28)...(28): LNA C is 5-methyl C
  misc_feature (30)...(30): LNA C is 5-methyl C
  misc_feature (32)...(32): LNA C is 5-methyl C
  misc_feature (34)...(34): LNA C is 5-methyl C
  misc_feature (36)...(36): LNA C is 5-methyl C
  misc_feature (38)...(38): LNA C is 5-methyl C
  misc_feature (40)...(40): LNA C is 5-methyl C

SEQ ID NO: 29 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4 conjugate
  misc_feature (1)...(40): LNA nucleoside
  misc_feature (2)...(2): LNA C is 5-methyl C
  misc_feature (4)...(4): LNA C is 5-methyl C
  misc_feature (6)...(6): LNA C is 5-methyl C
  misc_feature (8)...(8): LNA C is 5-methyl C
  misc_feature (10)...(10): LNA C is 5-methyl C
  misc_feature (12)...(12): LNA C is 5-methyl C
  misc_feature (14)...(14): LNA C is 5-methyl C
  misc_feature (16)...(16): LNA C is 5-methyl C
  misc_feature (18)...(18): LNA C is 5-methyl C
  misc_feature (20)...(20): LNA C is 5-methyl C
  misc_feature (22)...(22): LNA C is 5-methyl C
  misc_feature (24)...(24): LNA C is 5-methyl C
  misc_feature (26)...(26): LNA C is 5-methyl C
  misc_feature (28)...(28): LNA C is 5-methyl C
  misc_feature (30)...(30): LNA C is 5-methyl C
  misc_feature (32)...(32): LNA C is 5-methyl C
  misc_feature (34)...(34): LNA C is 5-methyl C
  misc_feature (36)...(36): LNA C is 5-methyl C
  misc_feature (38)...(38): LNA C is 5-methyl C
  misc_feature (40)...(40): LNA C is 5-methyl C

SEQ ID NO: 30 - DNA, 40 bp, Artificial Sequence; identical to SEQ ID NO: 1
  Modified Oligonucleotide
  misc_feature (1)...(40): phosphorothioate internucleoside linkages
  misc_feature (1)...(40): LNA nucleoside
  misc_feature (2)...(2): LNA C is 5-methyl C
  misc_feature (4)...(4): LNA C is 5-methyl C
  misc_feature (6)...(6): LNA C is 5-methyl C
  misc_feature (8)...(8): LNA C is 5-methyl C
  misc_feature (10)...(10): LNA C is 5-methyl C
  misc_feature (12)...(12): LNA C is 5-methyl C
  misc_feature (14)...(14): LNA C is 5-methyl C
  misc_feature (16)...(16): LNA C is 5-methyl C
  misc_feature (18)...(18): LNA C is 5-methyl C
  misc_feature (20)...(20): LNA C is 5-methyl C
  misc_feature (22)...(22): LNA C is 5-methyl C
  misc_feature (24)...(24): LNA C is 5-methyl C
  misc_feature (26)...(26): LNA C is 5-methyl C
  misc_feature (28)...(28): LNA C is 5-methyl C
  misc_feature (30)...(30): LNA C is 5-methyl C
  misc_feature (32)...(32): LNA C is 5-methyl C
  misc_feature (34)...(34): LNA C is 5-methyl C
  misc_feature (36)...(36): LNA C is 5-methyl C
  misc_feature (38)...(38): LNA C is 5-methyl C
  misc_feature (40)...(40): LNA C is 5-methyl C
  misc_feature (40)...(40): 3 prime poCpoApo-GalNac2 conjugate

SEQ ID NO: 31 - DNA, 40 bp, Artificial Sequence; identical to SEQ ID NO: 1
  Modified Oligonucleotide
  misc_feature (1)...(40): phosphorothioate internucleoside linkages
  misc_feature (1)...(40): LNA nucleoside
  misc_feature (1)...(1): 5 prime GalNac2-poCpoApo conjugate
  misc_feature (2)...(2): LNA C is 5-methyl C
  misc_feature (4)...(4): LNA C is 5-methyl C
  misc_feature (6)...(6): LNA C is 5-methyl C
  misc_feature (8)...(8): LNA C is 5-methyl C
  misc_feature (10)...(10): LNA C is 5-methyl C
  misc_feature (12)...(12): LNA C is 5-methyl C
  misc_feature (14)...(14): LNA C is 5-methyl C
  misc_feature (16)...(16): LNA C is 5-methyl C
  misc_feature (18)...(18): LNA C is 5-methyl C
  misc_feature (20)...(20): LNA C is 5-methyl C
  misc_feature (22)...(22): LNA C is 5-methyl C
  misc_feature (24)...(24): LNA C is 5-methyl C
  misc_feature (26)...(26): LNA C is 5-methyl C
  misc_feature (28)...(28): LNA C is 5-methyl C
  misc_feature (30)...(30): LNA C is 5-methyl C
  misc_feature (32)...(32): LNA C is 5-methyl C
  misc_feature (34)...(34): LNA C is 5-methyl C
  misc_feature (36)...(36): LNA C is 5-methyl C
  misc_feature (38)...(38): LNA C is 5-methyl C
  misc_feature (40)...(40): LNA C is 5-methyl C

SEQ ID NO: 32 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 33 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poApoApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 34 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poApoApoApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 35 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-poApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 36 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-poApoApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 37 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-poApoApoApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 38 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-porApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 39 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-porAporApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 40 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-porAporAporApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 41 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-porApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 42 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-porAporApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 43 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-porAporAporApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 44 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-pomApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 45 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-pomApomApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 46 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-pomApomApomApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 47 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-pomApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 48 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-pomApomApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 49 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-pomApomApomApo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 50 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poHEGpo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 51 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poHEGpoHEGpo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 52 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-poHEGpo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 53 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac6-poHEGpoHEGpo conjugate
  misc_feature (1)...(1): mA is 2′-O-Methyladenosine
  misc_feature (2)...(2): LNA nucleoside, LNA C is 5-methyl C
  misc_feature (3)...(3): mA is 2′-O-Methyladenosine
  misc_feature (4)...(4): LNA nucleoside, LNA C is 5-methyl C
  misc_feature (5)...(5): mA is 2′-O-Methyladenosine
  misc_feature (6)...(6): LNA nucleoside, LNA C is 5-methyl C
  misc_feature (7)...(7): mA is 2′-O-Methyladenosine
  misc_feature (8)...(8): LNA nucleoside, LNA C is 5-methyl C
  misc_feature (9)...(9): mA is 2′-O-Methyladenosine
  misc_feature (10)...(10): LNA nucleoside, LNA C is 5-methyl C
  misc_feature (11)...(11): rA is ribo-adenosine
  misc_feature (12)...(12): LNA nucleoside, LNA C is 5-methyl C
  misc_feature (13)...(13): mA is 2′-O-Methyladenosine
  misc_feature (14)...(14): LNA nucleoside, LNA C is 5-methyl C
  misc_feature (15)...(15): mA is 2′-O-Methyladenosine
  misc_feature (16)...(16): LNA nucleoside, LNA C is 5-methyl C
  misc_feature (17)...(17): rA is ribo-adenosine
  misc_feature (18)...(18): LNA nucleoside, LNA C is 5-methyl C
  misc_feature (19)...(19): mA is 2′-O-Methyladenosine
  misc_feature (20)...(20): LNA nucleoside, LNA C is 5-methyl C
  misc_feature (21)...(21): mA is 2′-O-Methyladenosine
  misc_feature (22)...(22): LNA nucleoside, LNA C is 5-methyl C
  misc_feature (23)...(23): rA is ribo-adenosine
  misc_feature (24)...(24): LNA nucleoside, LNA C is 5-methyl C
  misc_feature (25)...(25): mA is 2′-O-Methyladenosine
  misc_feature (26)...(26): LNA nucleoside, LNA C is 5-methyl C
  misc_feature (27)...(27): mA is 2′-O-Methyladenosine
  misc_feature (28)...(28): LNA nucleoside, LNA C is 5-methyl C
  misc_feature (29)...(29): rA is ribo-adenosine
  misc_feature (30)...(30): LNA nucleoside, LNA C is 5-methyl C
  misc_feature (31)...(31): mA is 2′-O-Methyladenosine
  misc_feature (32)...(32): LNA nucleoside, LNA C is 5-methyl C
  misc_feature (33)...(33): mA is 2′-O-Methyladenosine
  misc_feature (34)...(34): LNA nucleoside, LNA C is 5-methyl C
  misc_feature (35)...(35): rA is ribo-adenosine
  misc_feature (36)...(36): LNA nucleoside, LNA C is 5-methyl C
  misc_feature (37)...(37): mA is 2′-O-Methyladenosine
  misc_feature (38)...(38): LNA nucleoside, LNA C is 5-methyl C
  misc_feature (39)...(39): mA is 2′-O-Methyladenosine
  misc_feature (40)...(40): LNA nucleoside, LNA C is 5-methyl C

SEQ ID NO: 54 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4 conjugate
  misc_feature (1)...(1): mA is 2′-O-Methyladenosine
  misc_feature (2)...(2): m(5m)C is 2′-0, 5- dimethylcytidine
  misc_feature (3)...(3): mA is 2′-O-Methyladenosine
  misc_feature (4)...(4): m(5m)C is 2′-0, 5- dimethylcytidine
  misc_feature (5)...(5): mA is 2′-O-Methyladenosine
  misc_feature (6)...(6): m(5m)C is 2′-0, 5- dimethylcytidine
  misc_feature (7)...(7): mA is 2′-O-Methyladenosine
  misc_feature (8)...(8): m(5m)C is 2′-0, 5- dimethylcytidine
  misc_feature (9)...(9): mA is 2′-O-Methyladenosine
  misc_feature (10)...(10): m(5m)C is 2′-0, 5- dimethylcytidine
  misc_feature (11)...(11): rA is ribo-adenosine
  misc_feature (12)...(12): m(5m)C is 2′-0, 5- dimethylcytidine
  misc_feature (13)...(13): mA is 2′-O-Methyladenosine
  misc_feature (14)...(14): m(5m)C is 2′-0, 5- dimethylcytidine
  misc_feature (15)...(15): mA is 2′-O-Methyladenosine
  misc_feature (16)...(16): m(5m)C is 2′-0, 5- dimethylcytidine
  misc_feature (17)...(17): mA is 2′-O-Methyladenosine
  misc_feature (18)...(18): m(5m)C is 2′-0, 5- dimethylcytidine
  misc_feature (19)...(19): mA is 2′-O-Methyladenosine
  misc_feature (20)...(20): m(5m)C is 2′-0, 5- dimethylcytidine
  misc_feature (21)...(21): rA is ribo-adenosine
  misc_feature (22)...(22): m(5m)C is 2′-0, 5- dimethylcytidine
  misc_feature (23)...(23): mA is 2′-O-Methyladenosine
  misc_feature (24)...(24): m(5m)C is 2′-0, 5- dimethylcytidine
  misc_feature (25)...(25): mA is 2′-O-Methyladenosine
  misc_feature (26)...(26): m(5m)C is 2′-0, 5- dimethylcytidine
  misc_feature (27)...(27): mA is 2′-O-Methyladenosine
  misc_feature (28)...(28): m(5m)C is 2′-0, 5- dimethylcytidine
  misc_feature (29)...(29): mA is 2′-O-Methyladenosine
  misc_feature (30)...(30): m(5m)C is 2′-0, 5- dimethylcytidine
  misc_feature (31)...(31): rA is ribo-adenosine
  misc_feature (32)...(32): m(5m)C is 2′-0, 5- dimethylcytidine
  misc_feature (33)...(33): mA is 2′-O-Methyladenosine
  misc_feature (34)...(34): m(5m)C is 2′-0, 5- dimethylcytidine
  misc_feature (35)...(35): mA is 2′-O-Methyladenosine
  misc_feature (36)...(36): m(5m)C is 2′-0, 5- dimethylcytidine
  misc_feature (37)...(37): mA is 2′-O-Methyladenosine
  misc_feature (38)...(38): m(5m)C is 2′-0, 5- dimethylcytidine
  misc_feature (39)...(39): mA is 2′-O-Methyladenosine
  misc_feature (40)...(40): m(5m)C is 2′-0, 5- dimethylcytidine

SEQ ID NO: 55 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poApoApoApo conjugate
  misc_feature (1)...(1): mA is 2′-O-Methyladenosine
  misc_feature (2)...(2): m(5m)C is 2′-0, 5- dimethylcytidine
  misc_feature (3)...(3): mA is 2′-O-Methyladenosine
  misc_feature (4)...(4): m(5m)C is 2′-0, 5- dimethylcytidine
  misc_feature (5)...(5): mA is 2′-O-Methyladenosine
  misc_feature (6)...(6): m(5m)C is 2′-0, 5- dimethylcytidine
  misc_feature (7)...(7): mA is 2′-O-Methyladenosine
  misc_feature (8)...(8): m(5m)C is 2′-0, 5- dimethylcytidine
  misc_feature (9)...(9): mA is 2′-O-Methyladenosine
  misc_feature (10)...(10): m(5m)C is 2′-0, 5- dimethylcytidine
  misc_feature (11)...(11): rA is ribo-adenosine
  misc_feature (12)...(12): m(5m)C is 2′-0, 5- dimethylcytidine
  misc_feature (13)...(13): mA is 2′-O-Methyladenosine
  misc_feature (14)...(14): m(5m)C is 2′-0, 5- dimethylcytidine
  misc_feature (15)...(15): mA is 2′-O-Methyladenosine
  misc_feature (16)...(16): m(5m)C is 2′-0, 5- dimethylcytidine
  misc_feature (17)...(17): mA is 2′-O-Methyladenosine
  misc_feature (18)...(18): m(5m)C is 2′-0, 5- dimethylcytidine
  misc_feature (19)...(19): mA is 2′-O-Methyladenosine
  misc_feature (20)...(20): m(5m)C is 2′-0, 5- dimethylcytidine
  misc_feature (21)...(21): rA is ribo-adenosine
  misc_feature (22)...(22): m(5m)C is 2′-0, 5- dimethylcytidine
  misc_feature (23)...(23): mA is 2′-O-Methyladenosine
  misc_feature (24)...(24): m(5m)C is 2′-0, 5- dimethylcytidine
  misc_feature (25)...(25): mA is 2′-O-Methyladenosine
  misc_feature (26)...(26): m(5m)C is 2′-0, 5- dimethylcytidine
  misc_feature (27)...(27): mA is 2′-O-Methyladenosine
  misc_feature (28)...(28): m(5m)C is 2′-0, 5- dimethylcytidine
  misc_feature (29)...(29): mA is 2′-O-Methyladenosine
  misc_feature (30)...(30): m(5m)C is 2′-0, 5- dimethylcytidine
  misc_feature (31)...(31): rA is ribo-adenosine
  misc_feature (32)...(32): m(5m)C is 2′-0, 5- dimethylcytidine
  misc_feature (33)...(33): mA is 2′-O-Methyladenosine
  misc_feature (34)...(34): m(5m)C is 2′-0, 5- dimethylcytidine
  misc_feature (35)...(35): mA is 2′-O-Methyladenosine
  misc_feature (36)...(36): m(5m)C is 2′-0, 5- dimethylcytidine
  misc_feature (37)...(37): mA is 2′-O-Methyladenosine
  misc_feature (38)...(38): m(5m)C is 2′-0, 5- dimethylcytidine
  misc_feature (39)...(39): mA is 2′-O-Methyladenosine
  misc_feature (40)...(40): m(5m)C is 2′-0, 5- dimethylcytidine

SEQ ID NO: 56 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4 conjugate
  misc_feature (1)...(1): lnA is LNA adenosine
  misc_feature (2)...(2): m(5m)C is 2′-0, 5- dimethylcytidine
  misc_feature (3)...(3): mA is 2′-O-Methyladenosine
  misc_feature (4)...(4): m(5m)C is 2′-0, 5- dimethylcytidine
  misc_feature (5)...(5): mA is 2′-O-Methyladenosine
  misc_feature (6)...(6): m(5m)C is 2′-0, 5- dimethylcytidine
  misc_feature (7)...(7): mA is 2′-O-Methyladenosine
  misc_feature (8)...(8): m(5m)C is 2′-0, 5- dimethylcytidine
  misc_feature (9)...(9): mA is 2′-O-Methyladenosine
  misc_feature (10)...(10): m(5m)C is 2′-0, 5- dimethylcytidine
  misc_feature (11)...(11): rA is ribo-adenosine
  misc_feature (12)...(12): m(5m)C is 2′-0, 5- dimethylcytidine
  misc_feature (13)...(13): mA is 2′-O-Methyladenosine
  misc_feature (14)...(14): m(5m)C is 2′-0, 5- dimethylcytidine
  misc_feature (15)...(15): mA is 2′-O-Methyladenosine
  misc_feature (16)...(16): m(5m)C is 2′-0, 5- dimethylcytidine
  misc_feature (17)...(17): mA is 2′-O-Methyladenosine
  misc_feature (18)...(18): m(5m)C is 2′-0, 5- dimethylcytidine
  misc_feature (19)...(19): mA is 2′-O-Methyladenosine
  misc_feature (20)...(20): m(5m)C is 2′-0, 5- dimethylcytidine
  misc_feature (21)...(21): rA is ribo-adenosine
  misc_feature (22)...(22): m(5m)C is 2′-0, 5- dimethylcytidine
  misc_feature (23)...(23): mA is 2′-O-Methyladenosine
  misc_feature (24)...(24): m(5m)C is 2′-0, 5- dimethylcytidine
  misc_feature (25)...(25): mA is 2′-O-Methyladenosine
  misc_feature (26)...(26): m(5m)C is 2′-0, 5- dimethylcytidine
  misc_feature (27)...(27): mA is 2′-O-Methyladenosine
  misc_feature (28)...(28): m(5m)C is 2′-0, 5- dimethylcytidine
  misc_feature (29)...(29): mA is 2′-O-Methyladenosine
  misc_feature (30)...(30): m(5m)C is 2′-0, 5- dimethylcytidine
  misc_feature (31)...(31): rA is ribo-adenosine
  misc_feature (32)...(32): m(5m)C is 2′-0, 5- dimethylcytidine
  misc_feature (33)...(33): mA is 2′-O-Methyladenosine
  misc_feature (34)...(34): m(5m)C is 2′-0, 5- dimethylcytidine
  misc_feature (35)...(35): mA is 2′-O-Methyladenosine
  misc_feature (36)...(36): m(5m)C is 2′-0, 5- dimethylcytidine
  misc_feature (37)...(37): mA is 2′-O-Methyladenosine
  misc_feature (38)...(38): m(5m)C is 2′-0, 5- dimethylcytidine
  misc_feature (39)...(39): mA is 2′-O-Methyladenosine
  misc_feature (40)...(40): LNA nucleoside, LNA C is 5-methyl C

SEQ ID NO: 57 - DNA, 40 bp, Artificial Sequence; identical to SEQ ID NO: 1
  Modified Oligonucleotide
  misc_feature (1)...(40): phosphorothioate internucleoside linkages
  misc_feature (1)...(1): 5 prime GalNac4-poApoApoApo conjugate
  misc_feature (1)...(1): lnA is LNA adenosine
  misc_feature (2)...(2): m(5m)C is 2′-0, 5- dimethylcytidine
  misc_feature (3)...(3): mA is 2′-O-Methyladenosine
  misc_feature (4)...(4): m(5m)C is 2′-0, 5- dimethylcytidine
  misc_feature (5)...(5): mA is 2′-O-Methyladenosine
  misc_feature (6)...(6): m(5m)C is 2′-0, 5- dimethylcytidine
  misc_feature (7)...(7): mA is 2′-O-Methyladenosine
  misc_feature (8)...(8): m(5m)C is 2′-0, 5- dimethylcytidine
  misc_feature (9)...(9): mA is 2′-O-Methyladenosine
  misc_feature (10)...(10): m(5m)C is 2′-0, 5- dimethylcytidine
  misc_feature (11)...(11): rA is ribo-adenosine
  misc_feature (12)...(12): m(5m)C is 2′-0, 5- dimethylcytidine
  misc_feature (13)...(13): mA is 2′-O-Methyladenosine
  misc_feature (14)...(14): m(5m)C is 2′-0, 5- dimethylcytidine
  misc_feature (15)...(15): mA is 2′-O-Methyladenosine
  misc_feature (16)...(16): m(5m)C is 2′-0, 5- dimethylcytidine
  misc_feature (17)...(17): mA is 2′-O-Methyladenosine
  misc_feature (18)...(18): m(5m)C is 2′-0, 5- dimethylcytidine
  misc_feature (19)...(19): mA is 2′-O-Methyladenosine
  misc_feature (20)...(20): m(5m)C is 2′-0, 5- dimethylcytidine
  misc_feature (21)...(21): rA is ribo-adenosine
  misc_feature (22)...(22): m(5m)C is 2′-0, 5- dimethylcytidine
  misc_feature (23)...(23): mA is 2′-O-Methyladenosine
  misc_feature (24)...(24): m(5m)C is 2′-0, 5- dimethylcytidine
  misc_feature (25)...(25): mA is 2′-O-Methyladenosine
  misc_feature (26)...(26): m(5m)C is 2′-0, 5- dimethylcytidine
  misc_feature (27)...(27): mA is 2′-O-Methyladenosine
  misc_feature (28)...(28): m(5m)C is 2′-0, 5- dimethylcytidine
  misc_feature (29)...(29): mA is 2′-O-Methyladenosine
  misc_feature (30)...(30): m(5m)C is 2′-0, 5- dimethylcytidine
  misc_feature (31)...(31): rA is ribo-adenosine
  misc_feature (32)...(32): m(5m)C is 2′-0, 5- dimethylcytidine
  misc_feature (33)...(33): mA is 2′-O-Methyladenosine
  misc_feature (34)...(34): m(5m)C is 2′-0, 5- dimethylcytidine
  misc_feature (35)...(35): mA is 2′-O-Methyladenosine
  misc_feature (36)...(36): m(5m)C is 2′-0, 5- dimethylcytidine
  misc_feature (37)...(37): mA is 2′-O-Methyladenosine
  misc_feature (38)...(38): m(5m)C is 2′-0, 5- dimethylcytidine
  misc_feature (39)...(39): mA is 2′-O-Methyladenosine
  misc_feature (40)...(40): LNA nucleoside, LNA C is 5-methyl C